(19) United States
(12) Patent Application Publication
(10) Pub. No.: US 2025/0332098 A1
(43) Pub. Date: Oct. 30, 2025

(54) Formulations of DR5 Binding Polypeptides

(71) Applicant: Inhibrx Biosciences, Inc., La Jolla, CA (US)

(72) Inventors: Ashraf Amanullah, Carlsbad, CA (US); Brian Lobo, Clarksburg, MD (US)

(73) Assignee: Inhibrx Biosciences, Inc., La Jolla, CA (US)

(21) Appl. No.: 18/264,475

(22) PCT Filed: Feb. 18, 2022
(86) PCT No.: PCT/US2022/016935

§ 371 Date: Aug. 7, 2023

Related U.S. Application Data
(60) Provisional application No. 63/151,131, filed on Feb. 19, 2021.

(51) Int. Cl. A61K 9/08; A61K 9/19; A61K 47/10; A61K 47/20; A61K 47/22; A61K 47/26; C07K 16/28

(52) U.S. Cl. CPC A61K 9/08; A61K 9/19; A61K 47/10; A61K 47/20; A61K 47/22; A61K 47/26; C07K 16/2878; C07K 2317/24; C07K 2317/35; C07K 2317/94

(57) ABSTRACT

Provided herein are formulations of DR5-binding polypeptides. Uses of the DR5-binding polypeptides are also provided.

### CROSS-REFERENCE TO RELATED APPLICATIONS

This application claims the benefit of priority of U.S. Provisional Application No. 63/151,131, filed Feb. 19, 2021, which is incorporated by reference herein in its entirety for any purpose.

### SEQUENCE LISTING

This application contains a sequence listing submitted in electronic format. The file name is “2022-01-14_01202-0049-00PCT_Seq_List_ST25,” it was created on Jan. 14, 2022, and is 17,695 bytes in size.

### FIELD

The present invention relates to formulations of DR5-binding proteins, and methods of using the formulations. Such methods include, but are not limited to, methods of treating cancer.

### BACKGROUND

DR5 is a member of the TNF receptor superfamily (TNFRSF) and a cell surface receptor of the TNF-receptor superfamily that binds TNF-related apoptosis-inducing ligand (TRAIL). TRAIL evolved to play critical roles in mammalian development and host defense by selectively eradicating unwanted, infected and malignant cells from healthy cell populations. On binding the TNF receptor family members DR4 or DR5, TRAIL induces cell death via caspase-dependent apoptosis. DR5 appears to be the primary receptor on tumor cells that facilitates the observed tumor biased activity of the TRAIL pathway. DR5 is activated by the natural ligand TRAIL, which brings three DR5 receptors within close proximity thereby activating intracellular caspase-8 and initiating activation of other death-inducing caspases, such as caspases-9 and caspases-3. Thus, initiation of this cell death pathway requires clustering of DR5 receptors for efficient cell death.

### SUMMARY

Provided herein are stable liquid and lyophilized pharmaceutical formulations of multivalent DR5-binding polypeptides that are capable of agonizing DR5 signaling mediating direct cell death, and the use of the pharmaceutical formulations for treating cancer.

Embodiment 1. A pharmaceutical formulation comprising a DR5-binding polypeptide, wherein the formulation comprises 20-70 mg/mL DR5-binding polypeptide, 5-20 mM histidine, 7-10% w/v sucrose, and 0.1-0.8% poloxamer P188 at a pH of 5.3-6.7; and wherein the DR5-binding polypeptide comprises at least one VHH domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.

Embodiment 2. The pharmaceutical formulation of embodiment 1, wherein the formulation comprises 30-60 mg/mL DR5-binding polypeptide.

Embodiment 3. The pharmaceutical formulation of embodiment 1, wherein the formulation comprises 50 mg/mL DR5-binding polypeptide.

Embodiment 4. The pharmaceutical formulation of any one of embodiments 1-3, wherein the formulation comprises 7-15 mM histidine.

Embodiment 5. The pharmaceutical formulation of any one of embodiments 1-3, wherein the formulation comprises 10 mM histidine.

Embodiment 6. The pharmaceutical formulation of any one of embodiments 1-5, wherein the histidine is histidine HCl.

Embodiment 7. The pharmaceutical formulation of any one of embodiments 1-6, wherein the formulation comprises 8-9% sucrose.

Embodiment 8. The pharmaceutical formulation of any one of embodiments 1-7, wherein the formulation comprises 8% sucrose or 9% sucrose.

Embodiment 9. The pharmaceutical formulation of any one of embodiments 1-8, wherein the formulation comprises 0.2-0.4% poloxamer P188.

Embodiment 10. The pharmaceutical formulation of any one of embodiments 1-9, wherein the formulation comprises 0.2% poloxamer P188.

Embodiment 11. The pharmaceutical formulation of any one of embodiments 1-10, wherein the formulation comprises 1-10 mM, 2-8 mM, 3-7 mM, or 4-6 mM methionine.

Embodiment 12. The pharmaceutical formulation of any one of embodiments 1-11, wherein the formulation comprises 5 mM methionine.

Embodiment 13. The pharmaceutical formulation of any one of embodiments 1-12, wherein the pH of the formulation is 5.4-6.6, 5.5-6.5, 5.6-6.4, 5.7-6.3, or 5.8-6.2.

Embodiment 14. The pharmaceutical formulation of any one of embodiments 1-13, wherein the pH of the formulation is about 6.

Embodiment 15. The pharmaceutical formulation of any one of embodiments 1-14, wherein the formulation comprises 50 mg/mL DR5-binding polypeptide, 10 mM histidine HCl, 8% sucrose, and 0.2% poloxamer P188, and wherein the pH of the formulation is about 6.

Embodiment 16. The pharmaceutical formulation of embodiment 15, wherein the formulation consists essentially of 50 mg/mL DR5-binding polypeptide, 10 mM histidine HCl, 8% sucrose, 0.2% poloxamer P188, and water, and wherein the pH of the formulation is about 6.

Embodiment 17. The pharmaceutical formulation of any one of embodiments 1-16, wherein the DR5-binding polypeptide comprises a VHH domain comprising the amino acid sequence of SEQ ID NO: 4.

Embodiment 18. The pharmaceutical formulation of any one of embodiments 1-17, wherein the DR5-binding polypeptide comprises an Fc region.

Embodiment 19. The pharmaceutical formulation of embodiment 18, wherein the Fc region comprises the amino acid sequence of SEQ ID NO: 6.

Embodiment 20. The pharmaceutical formulation of any one of embodiments 1-19, wherein the DR5-binding polypeptide has the structure VHH-linker-VHH-linker-Fc.

Embodiment 21. The pharmaceutical formulation of embodiment 20, wherein the VHH-linker-VHH comprises the amino acid sequence of SEQ ID NO: 5.

Embodiment 22. The pharmaceutical formulation of any one of embodiments 1-21, wherein the DR5-binding polypeptide comprises the amino acid sequence of SEQ ID NO: 7.

Embodiment 23. The pharmaceutical formulation of any one of embodiments 1-21, wherein the DR5-binding polypeptide consists of the amino acid sequence of SEQ ID NO: 7.

Embodiment 24. A lyophilized formulation comprising a DR5-binding polypeptide, wherein the DR5-binding polypeptide comprises at least one VHH domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and wherein upon reconstitution of the lyophilized formulation in water to form an aqueous formulation, the aqueous formulation comprises 20-70 mg/mL DR5-binding polypeptide, 5-20 mM histidine, 7-10% sucrose, and 0.1-0.5% poloxamer P188, pH 5.3-6.7.

Embodiment 25. The lyophilized formulation of embodiment 24, wherein upon reconstitution of the formulation in water to form an aqueous formulation, the aqueous formulation comprises 30-60 mg/mL DR5-binding polypeptide.

Embodiment 26. The lyophilized formulation of embodiment 24, wherein upon reconstitution of the formulation in water to form an aqueous formulation, the aqueous formulation comprises 50 mg/mL DR5-binding polypeptide.

Embodiment 27. The lyophilized formulation of any one of embodiments 24-26, wherein upon reconstitution of the formulation in water to form an aqueous formulation, the aqueous formulation comprises 7-15 mM histidine.

Embodiment 28. The lyophilized formulation of any one of embodiments 24-26, wherein upon reconstitution of the formulation in water to form an aqueous formulation, the aqueous formulation comprises 10 mM histidine.

Embodiment 29. The lyophilized formulation of any one of embodiments 24-28, wherein the histidine is histidine HCl.

Embodiment 30. The lyophilized formulation of any one of embodiments 24-29, wherein upon reconstitution of the lyophilized formulation in water to form an aqueous formulation, the aqueous formulation comprises 8-9% sucrose.

Embodiment 31. The lyophilized formulation of any one of embodiments 24-30, wherein upon reconstitution of the formulation in water to form an aqueous formulation, the aqueous formulation comprises 8% sucrose or 9% sucrose.

Embodiment 32. The lyophilized formulation of any one of embodiments 24-31, wherein upon reconstitution of the formulation in water to form an aqueous formulation, the aqueous formulation comprises 0.2-0.4% poloxamer P188.

Embodiment 33. The lyophilized formulation of any one of embodiments 24-32, wherein upon reconstitution of the formulation in water to form an aqueous formulation, the aqueous formulation comprises 0.2% poloxamer P188.

Embodiment 34. The lyophilized formulation of any one of embodiments 24-33, wherein upon reconstitution of the formulation in water to form an aqueous formulation, the aqueous formulation comprises 1-10 mM, 2-8 mM, 3-7 mM, or 4-6 mM methionine.

Embodiment 35. The lyophilized formulation of any one of embodiments 24-33, wherein upon reconstitution of the formulation in water to form an aqueous formulation, the aqueous formulation comprises 5 mM methionine.

Embodiment 36. The lyophilized formulation of any one of embodiments 24-35, wherein upon reconstitution of the formulation in water to form an aqueous formulation, the pH of the aqueous formulation is 5.4-6.6, 5.5-6.5, 5.6-6.4, 5.7-6.3, or 5.8-6.2.

Embodiment 37. The lyophilized formulation of any one of embodiments 24-35, wherein upon reconstitution of the formulation in water to form an aqueous formulation, the pH of the aqueous formulation is about 6.

Embodiment 38. The lyophilized formulation of any one of embodiments 24-37, wherein upon reconstitution of the formulation in water to form an aqueous formulation, the aqueous formulation comprises 50 mg/mL DR5-binding polypeptide, 10 mM histidine HCl, 8% sucrose, and 0.2% poloxamer P188, pH about 6.

Embodiment 39. The lyophilized formulation of embodiment 28, wherein upon reconstitution of the formulation in water to form an aqueous formulation, the aqueous formulation consists essentially of 50 mg/mL DR5-binding polypeptide, 10 mM histidine HCl, 8% sucrose, 0.2% poloxamer P188, and water, and wherein the pH of the formulation is about 6.

Embodiment 40. The lyophilized formulation of any one of embodiments 24-39, wherein the DR5-binding polypeptide comprises a VHH domain comprising the amino acid sequence of SEQ ID NO: 4.

Embodiment 41. The lyophilized formulation of any one of embodiments 24-40, wherein the DR5-binding polypeptide comprises an Fc region.

Embodiment 42. The lyophilized formulation of embodiment 41, wherein the Fc region comprises the amino acid sequence of SEQ ID NO: 6.

Embodiment 43. The lyophilized formulation of any one of embodiments 24-42, wherein the DR5-binding polypeptide has the structure VHH-linker-VHH-linker-Fc.

Embodiment 44. The lyophilized formulation of embodiment 43, wherein the VHH-linker-VHH comprises the amino acid sequence of SEQ ID NO: 5.

Embodiment 45. The lyophilized formulation of any one of embodiments 24-44, wherein the DR5-binding polypeptide comprises the amino acid sequence of SEQ ID NO: 7.

Embodiment 46. The lyophilized formulation of any one of embodiments 24-44, wherein the DR5-binding polypeptide consists of the amino acid sequence of SEQ ID NO: 7.

Embodiment 47. A lyophilized formulation formed by lyophilizing the pharmaceutical formulation of any one of embodiments 1-23.

Embodiment 48. The lyophilized formulation of any one of embodiments 24-47, wherein following storage at 2-8° C. for up to 3 months, up to 6 months, up to 9 months, up to 12 months, or more than 12 months, the lyophilized formulation is an off-white, uniform and elegant cake without any visible impurities.

Embodiment 49. The lyophilized formulation of any one of embodiments 24-48, wherein following storage at 2-8° C. for up to 3 months, up to 6 months, up to 9 months, up to 12 months, or more than 12 months, upon reconstitution of the formulation in water to form an aqueous formulation, the aqueous formulation is practically free of visible particles.

Embodiment 50. The lyophilized formulation of any one of embodiments 24-49, wherein following storage at 2-8° C. for up to 3 months, up to 6 months, up to 9 months, up to 12 months, or more than 12 months, upon reconstitution of the formulation in water to form an aqueous formulation, less than 3% or less than 2% of the DR5-binding polypeptide present in the aqueous formulation is aggregated, as measured by size-exclusion chromatography.

Embodiment 51. The lyophilized formulation of embodiment 50, wherein less than 1% or less than 0.5% of the DR5-binding polypeptide present in the aqueous formulation is degraded, as measured by size-exclusion chromatography.

Embodiment 52. A pharmaceutical formulation formed by reconstituting the lyophilized formulation of any one of embodiments 24-51.

Embodiment 53. A method of treating cancer comprising administering to a subject with cancer the pharmaceutical formulation of any one of embodiments 1-23 and 52.

Embodiment 54. The method of embodiment 53, wherein the cancer is chondrosarcoma, mesothelioma, Ewing sarcoma, colorectal cancer, or pancreatic adenocarcinoma.

### DETAILED DESCRIPTION

Embodiments provided herein relate to formulations of DR5-binding polypeptides and their use in various methods of treating, for example, cancer.

### Definitions and Various Embodiments

The section headings used herein are for organizational purposes only and are not to be construed as limiting the subject matter described.

All references cited herein, including patent applications, patent publications, and Genbank Accession numbers are herein incorporated by reference, as if each individual reference were specifically and individually indicated to be incorporated by reference in its entirety.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., TRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993); and updated versions thereof.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

In general, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991). The “EU index as in Kabat” refers to the residue numbering of the human IgG1 EU antibody.

It is understood that embodiments of the invention described herein include “consisting” and/or “consisting essentially of” embodiments. As used herein, the singular form “a”, “an”, and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The phrase “reference sample”, “reference cell”, or “reference tissue”, denote a sample with at least one known characteristic that can be used as a comparison to a sample with at least one unknown characteristic. In some embodiments, a reference sample can be used as a positive or negative indicator. A reference sample can be used to establish a level of protein and/or mRNA that is present in, for example, healthy tissue, in contrast to a level of protein and/or mRNA present in the sample with unknown characteristics. In some embodiments, the reference sample comes from the same subject, but is from a different part of the subject than that being tested. In some embodiments, the reference sample is from a tissue area surrounding or adjacent to the cancer. In some embodiments, the reference sample is not from the subject being tested, but is a sample from a subject known to have, or not to have, a disorder in question (for example, a particular cancer or DR5-related disorder). In some embodiments, the reference sample is from the same subject, but from a point in time before the subject developed cancer. In some embodiments, the reference sample is from a benign cancer sample, from the same or a different subject. When a negative reference sample is used for comparison, the level of expression or amount of the molecule in question in the negative reference sample will indicate a level at which one of skill in the art will appreciate, given the present disclosure, that there is no and/or a low level of the molecule. When a positive reference sample is used for comparison, the level of expression or amount of the molecule in question in the positive reference sample will indicate a level at which one of skill in the art will appreciate, given the present disclosure, that there is a level of the molecule.

The terms “benefit”, “clinical benefit”, “responsiveness”, and “therapeutic responsiveness” as used herein in the context of benefiting from or responding to administration of a therapeutic agent, can be measured by assessing various endpoints, e.g., inhibition, to some extent, of disease progression, including slowing down and complete arrest; reduction in the number of disease episodes and/or symptoms; reduction in lesion size; inhibition (that is, reduction, slowing down or complete stopping) of disease cell infiltration into adjacent peripheral organs and/or tissues; inhibition (that is, reduction, slowing down or complete stopping) of disease spread; relief, to some extent, of one or more symptoms associated with the disorder; increase in the length of disease-free presentation following treatment, for example, progression-free survival; increased overall survival; higher response rate; and/or decreased mortality at a given point of time following treatment. A subject or cancer that is “non-responsive” or “fails to respond” is one that has failed to meet the above noted qualifications to be “responsive”.

The terms “nucleic acid molecule”, “nucleic acid” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides comprised in the nucleic acid molecule or polynucleotide.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The terms “DR5,” “death receptor 5,” and “TNFRSF10B” as used herein refer to any native, mature DR5 that results from processing of a DR5 precursor in a cell. The term includes DR5 from any vertebrate source, including mammals such as primates (e.g., humans and cynomolgus or rhesus monkeys) and rodents (e.g., mice and rats), unless otherwise indicated. The term also includes naturally-occurring variants of DR5, such as splice variants or allelic variants. A nonlimiting exemplary precursor human DR5 amino acid sequence is shown, e.g., in NCBI Accession No. NP_003833.4. See SEQ ID NO: 8. A nonlimiting exemplary precursor human DR5 amino acid sequence is shown, e.g., in SEQ ID NO: 9.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A single-domain antibody (sdAb) or VHH-containing polypeptide “specifically binds” or “preferentially binds” to a target if it binds with greater affinity, avidity, more readily, and/or with greater duration than it binds to other substances. For example, a sdAb or VHH-containing polypeptide that specifically or preferentially binds to a DR5 epitope is a sdAb or VHH-containing polypeptide that binds this epitope with greater affinity, avidity, more readily, and/or with greater duration than it binds to other DR5 epitopes or non-DR5 epitopes. It is also understood by reading this definition that; for example, a sdAb or VHH-containing polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

As used herein, the term “epitope” refers to a site on a target molecule (for example, an antigen, such as a protein, nucleic acid, carbohydrate or lipid) to which an antigen-binding molecule (for example, a sdAb or VHH-containing polypeptide) binds. Epitopes often include a chemically active surface grouping of molecules such as amino acids, polypeptides or sugar side chains and have specific three-dimensional structural characteristics as well as specific charge characteristics. Epitopes can be formed both from contiguous and/or juxtaposed noncontiguous residues (for example, amino acids, nucleotides, sugars, lipid moiety) of the target molecule. Epitopes formed from contiguous residues (for example, amino acids, nucleotides, sugars, lipid moiety) typically are retained on exposure to denaturing solvents whereas epitopes formed by tertiary folding typically are lost on treatment with denaturing solvents. An epitope may include but is not limited to at least 3, at least 5 or 8-10 residues (for example, amino acids or nucleotides). In some embodiments, an epitope is less than 20 residues (for example, amino acids or nucleotides) in length, less than 15 residues or less than 12 residues. Two antibodies may bind the same epitope within an antigen if they exhibit competitive binding for the antigen. In some embodiments, an epitope can be identified by a certain minimal distance to a CDR residue on the antigen-binding molecule. In some embodiments, an epitope can be identified by the above distance, and further limited to those residues involved in a bond (for example, a hydrogen bond) between a residue of the antigen-binding molecule and an antigen residue. An epitope can be identified by various scans as well, for example an alanine or arginine scan can indicate one or more residues that the antigen-binding molecule can interact with. Unless explicitly denoted, a set of residues as an epitope does not exclude other residues from being part of the epitope for a particular antigen-binding molecule. Rather, the presence of such a set designates a minimal series (or set of species) of epitopes. Thus, in some embodiments, a set of residues identified as an epitope designates a minimal epitope of relevance for the antigen, rather than an exclusive list of residues for an epitope on an antigen.

A “nonlinear epitope” or “conformational epitope” comprises noncontiguous polypeptides, amino acids and/or sugars within the antigenic protein to which an antigen-binding molecule specific to the epitope binds. In some embodiments, at least one of the residues will be noncontiguous with the other noted residues of the epitope; however, one or more of the residues can also be contiguous with the other residues.

A “linear epitope” comprises contiguous polypeptides, amino acids and/or sugars within the antigenic protein to which an antigen-binding molecule specific to the epitope binds. It is noted that, in some embodiments, not every one of the residues within the linear epitope need be directly bound (or involved in a bond) by the antigen-binding molecule. In some embodiments, linear epitopes can be from immunizations with a peptide that effectively consisted of the sequence of the linear epitope, or from structural sections of a protein that are relatively isolated from the remainder of the protein (such that the antigen-binding molecule can interact, at least primarily), just with that sequence section.

The term “antibody” is used in the broadest sense and encompass various polypeptides that comprise antibody-like antigen-binding domains, including but not limited to conventional antibodies (typically comprising at least one heavy chain and at least one light chain), single-domain antibodies (sdAbs, comprising at least one VHH domain and an Fc region), VHH-containing polypeptides (polypeptides comprising at least one VHH domain), and fragments of any of the foregoing so long as they exhibit the desired antigen-binding activity. In some embodiments, an antibody comprises a dimerization domain. Such dimerization domains include, but are not limited to, heavy chain constant domains (comprising CH1, hinge, CH2, and CH3, where CH1 typically pairs with a light chain constant domain, CL, while the hinge mediates dimerization) and Fc regions (comprising hinge, CH2, and CH3, where the hinge mediates dimerization).

The term antibody also includes, but is not limited to, chimeric antibodies, humanized antibodies, and antibodies of various species such as camelid (including llama), shark, mouse, human, cynomolgus monkey, etc.

The term “antigen-binding domain” as used herein refers to a portion of an antibody sufficient to bind antigen. In some embodiments, an antigen binding domain of a conventional antibody comprises three heavy chain CDRs and three light chain CDRs. Thus, in some embodiments, an antigen binding domain comprises a heavy chain variable region comprising CDR1-FR2-CDR2-FR3-CDR3, and any portions of FR1 and/or FR4 required to maintain binding to antigen, and a light chain variable region comprising CDR1-FR2-CDR2-FR3-CDR3, and any portions of FR1 and/or FR4 required to maintain binding to antigen. In some embodiments, an antigen-binding domain of an sdAb or VHH-containing polypeptide comprises three CDRs of a VHH domain. Thus, in some embodiments, an antigen binding domain of an sdAb or VHH-containing polypeptide comprises a VHH domain comprising CDR1-FR2-CDR2-FR3-CDR3, and any portions of FR1 and/or FR4 required to maintain binding to antigen.

The term “VHH” or “VHH domain” or “VHH antigen-binding domain” as used herein refers to the antigen-binding portion of a single-domain antibody, such as a camelid antibody or shark antibody. In some embodiments, a VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so long as the VHH substantially maintains antigen binding and specificity.

The terms “single domain antibody” and “sdAb” are used interchangeably herein to refer to an antibody comprising at least one monomeric domain, such as a VHH domain, without a light chain, and an Fc region. In some embodiments, an sdAb is a dimer of two polypeptides wherein each polypeptide comprises at least one VHH domain and an Fc region. As used herein, the terms “single domain antibody” and “sdAb” encompass polypeptides that comprise multiple VHH domains, such as a polypeptide having the structure VHH₁-VHH₂-Fc or VHH₁-VHH₂-VHH₃-Fc, wherein VHH₁, VHH₂, and VHH₃ may be the same or different.

The term “VHH-containing polypeptide” refers to a polypeptide that comprises at least one VHH domain. In some embodiments, a VHH polypeptide comprises two, three, or four or more VHH domains, wherein each VHH domain may be the same or different. In some embodiments, a VHH-containing polypeptide comprises an Fc region. In some such embodiments, the VHH-containing polypeptide may be referred to as an sdAb. Further, in some such embodiments, the VHH polypeptide may form a dimer. Nonlimiting structures of VHH-containing polypeptides, which are also sdAbs, include VHH₁-Fc, VHH₁-VHH₂-Fc, and VHH₁-VHH₂-VHH₃-Fc, wherein VHH₁, VHH₂, and VHH₃ may be the same or different. In some embodiments of such structures, one VHH may be connected to another VHH by a linker, or one VHH may be connected to the Fc by a linker. In some such embodiments, the linker comprises 1-20 amino acids, preferably 1-20 amino acids predominantly composed of glycine and, optionally, serine. In some embodiments, when a VHH-containing polypeptide comprises an Fc, it forms a dimer. Thus, the structure VHH₁-VHH₂-Fc, if it forms a dimer, is considered to be tetravalent (i.e., the dimer has four VHH domains). Similarly, the structure VHH₁-VHH₂-VHH₃-Fc, if it forms a dimer, is considered to be hexavalent (i.e., the dimer has six VHH domains).

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. Thus, a sample of monoclonal antibodies can bind to the same epitope on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3.

The term “heavy chain constant region” as used herein refers to a region comprising at least three heavy chain constant domains, C_{H}1, hinge, C_{H}2, and C_{H}3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a constant region is an IgM antibody, and an antibody comprising an F constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

A “Fc region” as used herein refers to a portion of a heavy chain constant region comprising CH2 and CH3. In some embodiments, an Fc region comprises a hinge, CH2, and CH3. In various embodiments, when an Fc region comprises a hinge, the hinge mediates dimerization between two Fc-containing polypeptides. An Fc region may be of any antibody heavy chain constant region isotype discussed herein. In some embodiments, an Fc region is an IgG1, IgG2, IgG3, or IgG4.

An “acceptor human framework” as used herein is a framework comprising the amino acid sequence of a heavy chain variable domain (V_{H}) framework derived from a human immunoglobulin framework or a human consensus framework, as discussed herein. An acceptor human framework derived from a human immunoglobulin framework or a human consensus framework can comprise the same amino acid sequence thereof, or it can contain amino acid sequence changes. In some embodiments, the number of amino acid changes are fewer than 10, or fewer than 9, or fewer than 8, or fewer than 7, or fewer than 6, or fewer than 5, or fewer than 4, or fewer than 3, across all of the human frameworks in a single antigen binding domain, such as a VHH.

“Affinity” refers to the strength of the sum total of noncovalent interactions between a single binding site of a molecule (for example, an antibody, such as an sdAb, or VHH-containing polypeptide) and its binding partner (for example, an antigen). The affinity or the apparent affinity of a molecule X for its partner Y can generally be represented by the dissociation constant (K_{D}) or the K_{D-apparent}, respectively. Affinity can be measured by common methods known in the art (such as, for example, ELISA K_{D}, KinExA, flow cytometry, and/or surface plasmon resonance devices), including those described herein. Such methods include, but are not limited to, methods involving BIAcore®, Octet®, or flow cytometry.

The term “K_{D}”, as used herein, refers to the equilibrium dissociation constant of an antigen-binding molecule/antigen interaction. When the term “K_{D}” is used herein, it includes K_{D} and K_{D-apparent}.

In some embodiments, the K_{D} of the antigen-binding molecule is measured by flow cytometry using an antigen-expressing cell line and fitting the mean fluorescence measured at each antibody concentration to a non-linear one-site binding equation (Prism Software graphpad). In some such embodiments, the K_{D} is K_{D-apparent}.

The term “biological activity” refers to any one or more biological properties of a molecule (whether present naturally as found in vivo, or provided or enabled by recombinant means). Biological properties include, but are not limited to, binding a ligand, inducing or increasing cell proliferation, and inducing or increasing expression of cytokines.

An “agonist” or “activating” antibody is one that increases and/or activates a biological activity of the target antigen. In some embodiments, the agonist antibody binds to an antigen and increases its biologically activity by at least about 20%, 40%, 60%, 80%, 85% or more.

An “antagonist”, a “blocking” or “neutralizing” antibody is one that inhibits, decreases and/or inactivates a biological activity of the target antigen. In some embodiments, the neutralizing antibody binds to an antigen and reduces its biologically activity by at least about 20%, 40%, 60%, 80%, 85% 90%, 95%, 99% or more.

An “affinity matured” sdAb or VHH-containing polypeptide refers to a sdAb or VHH-containing polypeptide with one or more alterations in one or more CDRs compared to a parent sdAb or VHH-containing polypeptide that does not possess such alterations, such alterations resulting in an improvement in the affinity of the sdAb or VHH-containing polypeptide for antigen.

A “humanized VHH” as used herein refers to a VHH in which one or more framework regions have been substantially replaced with human framework regions. In some instances, certain framework region (FR) residues of the human immunoglobulin are replaced by corresponding non-human residues. Furthermore, the humanized VHH can comprise residues that are found neither in the original VHH nor in the human framework sequences, but are included to further refine and optimize sdAb VHH-containing polypeptide performance. In some embodiments, a humanized sdAb or VHH-containing polypeptide comprises a human Fc region. As will be appreciated, a humanized sequence can be identified by its primary sequence and does not necessarily denote the process by which the antibody was created.

An “effector-positive Fc region” possesses an “effector function” of a native sequence Fc region. Exemplary “effector functions” include Fc receptor binding; Clq binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (See, for example, Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. For example, the term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, for example, Ghetie and Ward, Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some embodiments the two or more substantially similar values differ by no more than about any one of 5%, 10%, 15%, 20%, 25%, or 50%.

A polypeptide “variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved antigen binding, decreased immunogenicity, or improved ADCC or CDC.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” and “subject” are used interchangeably herein to refer to an animal; for example, a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder.

A “disease” or “disorder” as used herein refers to a condition where treatment is needed and/or desired.

The term “tumor cell”, “cancer cell”, “cancer”, “tumor”, and/or “neoplasm”, unless otherwise designated, are used herein interchangeably and refer to a cell (or cells) exhibiting an uncontrolled growth and/or abnormal increased cell survival and/or inhibition of apoptosis which interferes with the normal functioning of bodily organs and systems. Included in this definition are benign and malignant cancers, polyps, hyperplasia, as well as dormant tumors or micrometastases.

The terms “cancer” and “tumor” encompass solid and hematological/lymphatic cancers and also encompass malignant, pre-malignant, and benign growth, such as dysplasia. Exemplary cancers include, but are not limited to: basal cell carcinoma, biliary tract cancer; bladder cancer; bone cancer; brain and central nervous system cancer; breast cancer; cancer of the peritoneum; cervical cancer; choriocarcinoma; chondrosarcoma, Ewing sarcoma, colon and rectum cancer (colorectal cancer); connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer, such as pancreatic adenocarcinoma; prostate cancer; retinoblastoma; rhabdomyosarcoma; rectal cancer; cancer of the respiratory system; mesothelioma; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In some embodiments, an “increase” or “decrease” refers to a statistically significant increase or decrease, respectively. As will be clear to the skilled person, “modulating” can also involve effecting a change (which can either be an increase or a decrease) in affinity, avidity, specificity and/or selectivity of a target or antigen, for one or more of its ligands, binding partners, partners for association into a homomultimeric or heteromultimeric form, or substrates; effecting a change (which can either be an increase or a decrease) in the sensitivity of the target or antigen for one or more conditions in the medium or surroundings in which the target or antigen is present (such as pH, ion strength, the presence of co-factors, etc.); and/or cellular proliferation or cytokine production, compared to the same conditions but without the presence of a test agent. This can be determined in any suitable manner and/or using any suitable assay known per se or described herein, depending on the target involved.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

“Ameliorating” means a lessening or improvement of one or more symptoms as compared to not administering a therapeutic agent. “Ameliorating” also includes shortening or reduction in duration of a symptom.

The term “anti-cancer agent” is used herein in its broadest sense to refer to agents that are used in the treatment of one or more cancers. Exemplary classes of such agents in include, but are not limited to, chemotherapeutic agents, anti-cancer biologics (such as cytokines, receptor extracellular domain-Fc fusions, and antibodies), radiation therapy, CAR-T therapy, therapeutic oligonucleotides (such as antisense oligonucleotides and siRNAs) and oncolytic viruses.

The term “biological sample” means a quantity of a substance from a living thing or formerly living thing. Such substances include, but are not limited to, blood, (for example, whole blood), plasma, serum, urine, amniotic fluid, synovial fluid, endothelial cells, leukocytes, monocytes, other cells, organs, tissues, bone marrow, lymph nodes and spleen.

The term “control” or “reference” refers to a composition known to not contain an analyte (“negative control”) or to contain an analyte (“positive control”). A positive control can comprise a known concentration of analyte.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. Unless otherwise specified, the terms “reduce”, “inhibit”, or “prevent” do not denote or require complete prevention over all time, but just over the time period being measured.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “pharmaceutical formulation” and “pharmaceutical composition” are used interchangeably and refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.

Administration “in combination with” one or more further therapeutic agents includes simultaneous (concurrent) and sequential administration in any order.

The term “concurrently” is used herein to refer to administration of two or more therapeutic agents, where at least part of the administration overlaps in time, or where the administration of one therapeutic agent falls within a short period of time relative to administration of the other therapeutic agent, or wherein the therapeutic effects of both agents overlap for at least a period of time.

The term “sequentially” is used herein to refer to administration of two or more therapeutic agents that does not overlap in time, or wherein the therapeutic effects of the agents do not overlap.

As used herein, “in conjunction with” refers to administration of one treatment modality in addition to another treatment modality. As such, “in conjunction with” refers to administration of one treatment modality before, during, or after administration of the other treatment modality to the individual.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (for example, a package or container) or kit comprising at least one reagent, for example, a medicament for treatment of a disease or disorder (for example, cancer), or a probe for specifically detecting a biomarker described herein. In some embodiments, the manufacture or kit is promoted, distributed, or sold as a unit for performing the methods described herein.

The terms “label” and “detectable label” mean a moiety attached, for example, to an antibody or antigen to render a reaction (for example, binding) between the members of the specific binding pair, detectable. The labeled member of the specific binding pair is referred to as “detectably labeled.” Thus, the term “labeled binding protein” refers to a protein with a label incorporated that provides for the identification of the binding protein. In some embodiments, the label is a detectable marker that can produce a signal that is detectable by visual or instrumental means, for example, incorporation of a radiolabeled amino acid or attachment to a polypeptide of biotinyl moieties that can be detected by marked avidin (for example, streptavidin containing a fluorescent marker or enzymatic activity that can be detected by optical or colorimetric methods). Examples of labels for polypeptides include, but are not limited to, the following: radioisotopes or radionuclides (for example, ³H, ¹⁴C, ³⁵S, ⁹⁰Y ⁹⁹Tc, ¹¹¹In, ¹²⁵I, ¹³¹I, ¹⁷⁷Lu, ¹⁶⁶Ho, or ¹⁵³Sm); chromogens, fluorescent labels (for example, FITC, rhodamine, lanthanide phosphors), enzymatic labels (for example, horseradish peroxidase, luciferase, alkaline phosphatase); chemiluminescent markers; biotinyl groups; predetermined polypeptide epitopes recognized by a secondary reporter (for example, leucine zipper pair sequences, binding sites for secondary antibodies, metal binding domains, epitope tags); and magnetic agents, such as gadolinium chelates. Representative examples of labels commonly employed for immunoassays include moieties that produce light, for example, acridinium compounds, and moieties that produce fluorescence, for example, fluorescein. In this regard, the moiety itself may not be detectably labeled but may become detectable upon reaction with yet another moiety.

### Exemplary DR5-Binding Polypeptides

Provided herein are formulations of DR5-binding polypeptides. In various embodiments, a DR5-binding polypeptide comprises at least one VHH domain comprising CDR1 comprising the sequence of SEQ ID NO: 1, a CDR2 comprising the sequence of SEQ ID NO: 2, and a CDR3 comprising the sequence of SEQ ID NO: 3. In some embodiments, at least one VHH domain is humanized. In some embodiments, a DR5-binding polypeptide comprises at least one VHH domain comprising the amino acid sequence of SEQ ID NO: 4.

In some embodiments, a DR5-binding polypeptide comprises at least one VHH domain that binds DR5 and an Fc region. In some embodiments, a DR5-binding polypeptide provided herein comprises two VHH domains that bind DR5 and an Fc region. In some embodiments, an Fc region mediates dimerization of the DR5-binding polypeptide at physiological conditions such that a dimer is formed that doubles the number of DR5 binding sites. For example, a DR5-binding polypeptide comprising two VHH domains that bind DR5 and an Fc region is divalent as a monomer, but at physiological conditions, the Fc region may mediate dimerization, such that the DR5-binding polypeptide is a tetravalent dimer under such conditions.

In some embodiments, a DR5-binding polypeptide comprises the structure VHH-linker-VHH-linker-Fc. In some embodiments, the VHH-linker-VHH portion of the DR5-binding polypeptide comprises the amino acid sequence of SEQ ID NO: 5. In some embodiments, the Fc comprises a hinge. In some such embodiments, the Fc comprises the amino acid sequence of SEQ ID NO: 6. In some embodiments, the DR5-binding polypeptide comprises the amino acid sequence of SEQ ID NO: 7, which includes two VHH domains and an Fc region.

In some embodiments, a VHH domain that binds DR5 may be humanized. Humanized antibodies (such as sdAbs or VHH-containing polypeptides) are useful as therapeutic molecules because humanized antibodies reduce or eliminate the human immune response to non-human antibodies, which can result in an immune response to an antibody therapeutic, and decreased effectiveness of the therapeutic. Generally, a humanized antibody comprises one or more variable domains in which CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13: 1619-1633, and are further described, for example, in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling). Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272: 10678-10684 and Rosok et al., (1996) J. Biol. Chem. 271:22611-22618). Typically, the FR regions of a VHH are replaced with human FR regions to make a humanized VHH. In some embodiments, certain FR residues of the human FR are replaced in order to improve one or more properties of the humanized VHH. VHH domains with such replaced residues are still referred to herein as “humanized.”

In various embodiments, an Fc region included in a DR5-binding polypeptide is a human Fc region, or is derived from a human Fc region.

In some embodiments, an Fc region included in a DR5-binding polypeptide is derived from a human Fc region, and comprises a three amino acid deletion in the lower hinge corresponding to IgG1 E233, L234, and L235, herein referred to as “Fc xELL.” Fc xELL polypeptides do not engage FcγRs and thus are referred to as “effector silent” or “effector null”, however in some embodiments, xELL Fc regions bind FcRn and therefore have extended half-life and transcytosis associated with FcRn mediated recycling. In some embodiments, the Fc region is a human IgG1 xELL Fc region.

### Polypeptide Expression and Production

Nucleic acid molecules comprising polynucleotides that encode a DR5-binding polypeptide are provided. In some embodiments, the nucleic acid molecule may also encode a leader sequence that directs secretion of the DR5-binding polypeptide, which leader sequence is typically cleaved such that it is not present in the secreted polypeptide. The leader sequence may be a native heavy chain (or VHH) leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

Vectors comprising nucleic acids that encode the DR5-binding polypeptide described herein are provided. Such vectors include, but are not limited to, DNA vectors, phage vectors, viral vectors, retroviral vectors, etc. In some embodiments, a vector is selected that is optimized for expression of polypeptides in a desired cell type, such as CHO or CHO-derived cells, or in NSO cells. Exemplary such vectors are described, for example, in Running Deer et al., Biotechnol. Prog. 20:880-889 (2004).

In some embodiments, a DR5-binding polypeptide may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NSO cells. In some embodiments, the DR5-binding polypeptide may be expressed in yeast. See, e.g., U.S. Publication No. US 2006/0270045 A1. In some embodiments, a particular eukaryotic host cell is selected based on its ability to make desired post-translational modifications to the polypeptide. For example, in some embodiments, CHO cells produce polypeptides that have a higher level of sialylation than the same polypeptide produced in 293 cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the nucleic acids or vectors described herein are also provided. In some embodiments, a host cell that expresses a DR5-binding polypeptide described herein is provided. The DR5-binding polypeptides expressed in host cells can be purified by any suitable method. Such methods include, but are not limited to, the use of affinity matrices or hydrophobic interaction chromatography. Suitable affinity ligands include the ROR1 ECD and agents that bind Fc regions. For example, a Protein A, Protein G, Protein A/G, or an antibody affinity column may be used to bind the Fc region and to purify a DR5-binding polypeptide that comprises an Fc region. Hydrophobic interactive chromatography, for example, a butyl or phenyl column, may also suitable for purifying some polypeptides such as antibodies. Ion exchange chromatography (for example anion exchange chromatography and/or cation exchange chromatography) may also suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also suitable for purifying some polypeptides such as antibodies. Many methods of purifying polypeptides are known in the art.

In some embodiments, the DR5-binding polypeptide is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

In some embodiments, a DR5-binding polypeptide prepared by the methods described above are provided. In some embodiments, the DR5-binding polypeptide is prepared in a host cell. In some embodiments, the DR5-binding polypeptide is prepared in a cell-free system. In some embodiments, the DR5-binding polypeptide is purified. In some embodiments, a cell culture media comprising a DR5-binding polypeptide is provided.

In some embodiments, compositions comprising antibodies prepared by the methods described above are provided. In some embodiments, the composition comprises a DR5-binding polypeptide prepared in a host cell. In some embodiments, the composition comprises a DR5-binding polypeptide prepared in a cell-free system. In some embodiments, the composition comprises a purified DR5-binding polypeptide.

### Pharmaceutical Formulations of DR5-Binding Polypeptides

In some embodiments, pharmaceutical formulations of DR5-binding polypeptides are aqueous liquid formulations. In other embodiments, the formulations are lyophilized. In either case, the formulation comprises a DR5-binding polypeptide. In some embodiments, the DR5-binding polypeptide comprises at least one VHH domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3. In some embodiments, at least one VHH domain is humanized. In some embodiments, a DR5-binding polypeptide comprises at least one VHH domain comprising the amino acid sequence of SEQ ID NO: 4. In some embodiments, a DR5-binding polypeptide comprises the structure VHH-linker-VHH-linker-Fc. In some embodiments, the VHH-linker-VHH portion of the DR5-binding polypeptide comprises the amino acid sequence of SEQ ID NO: 5. In some embodiments, the Fc comprises a hinge. In some such embodiments, the Fc comprises the amino acid sequence of SEQ ID NO: 6. In some embodiments, the DR5-binding polypeptide comprises the amino acid sequence of SEQ ID NO: 7, which includes two VHH domains and an Fc region.

In some embodiments, the concentration of DR5-binding polypeptide in an aqueous formulation herein, or in a reconstitution of a lyophilized formulation as described herein is, for example, 20-70 mg/mL, such as 30-60 mg/mL, 20-60 mg/mL, 20-50 mg/mL, 20-40 mg/mL, 30-70 mg/mL, 30-50 mg/mL, 30-40 mg/mL, 50-70 mg/mL, or 50-60 mg/mL. In some embodiments, the formulation comprises 20 mg/mL, 30 mg/mL, 40 mg/mL, 50 mg/mL, 60 mg/mL, or 70 mg/mL DR5-binding polypeptide. In some embodiments, the formulation comprises 50 mg/mL DR5-binding polypeptide.

In some embodiments, the pH of the formulation is between 5.3 and 6.7, such as from 5.4-6.6, 5.5-6.5, 5.6-6.4, 5.6-6.5, 5.7-6.5, 5.8-6.5, 5.9-6.5, 6-6.5, 5.5-6.4, 5.5-6.3, 5.5-6.2, 5.5-6.1, 5.5-6, 5.8-6.2, 5.8-6, 5.9-6, 5.9-6.1, 6-6.1, or 6-6.2. In some embodiments, the pH of the formulation is about 5.5, about 5.6, about 5.7, about 5.8, about 5.9, about 6, about 6.1, about 6.2, about 6.3, about 6.4, or about 6.5. In some embodiments, the pH of the formulation is about 6. In some embodiments, the pH of the formulation is between 5.5 to 6.5.

In some embodiments, the formulation comprises histidine, and its concentration may be, for example, 5-20 mM histidine, such as 5-15 mM, 7-15 mM, 5-12 mM, 7-12 mM. In some embodiments, the formulation comprises 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM histidine. In some embodiments, the formulation comprises 10 mM histidine. In some embodiments, the histidine is histidine HCl.

In some embodiments, the formulation comprises sucrose, and its concentration may be, for example, 7-10% w/v, such as 7-9%, 8-10%, 8-9%, 7%, 8%, 9%, or 10% w/v. In some embodiments, the formulation comprises 8% sucrose. In some embodiments, the formulation comprises 9% sucrose.

In some embodiments, the formulation comprises poloxamer 188 (P188), and its concentration may be, for example, 0.1-0.8%, such as 0.1-0.5%, such as 0.2-0.4%, 0.2-0.5%, 0.3-0.5%, 0.3-0.4%, 0.1-0.2%, 0.1%, 0.2%, 0.3%, 0.4%, 0.5%, 0.6%, 0.7%, or 0.8%. In some embodiments, the formulation comprises 0.2% poloxamer P188.

A nonlimiting exemplary formulation comprises 50 mg/mL DR5-binding polypeptide, 10 mM histidine HCl, 8% sucrose, and 0.2% poloxamer P188, and wherein the pH of the formulation is about 6. In some embodiments, the DR5-binding polypeptide comprises at least one VHH domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3. In some embodiments, at least one VHH domain is humanized. In some embodiments, a DR5-binding polypeptide comprises at least one VHH domain comprising the amino acid sequence of SEQ ID NO: 4. In some embodiments, a DR5-binding polypeptide comprises the structure VHH-linker-VHH-linker-Fc. In some embodiments, the VHH-linker-VHH portion of the DR5-binding polypeptide comprises the amino acid sequence of SEQ ID NO: 5. In some embodiments, the Fc comprises a hinge. In some such embodiments, the Fc comprises the amino acid sequence of SEQ ID NO: 6. In some embodiments, the DR5-binding polypeptide comprises the amino acid sequence of SEQ ID NO: 7, which includes two VHH domains and an Fc region.

In some embodiments, the pharmaceutical formulation of a DR5-binding polypeptide is provided in lyophilized form. Methods of lyophilizing protein-containing drug products are well known in the art. In some embodiments, a lyophilized formulation provided herein is stable for at least or up to 3 months, at least or up to 6 months, at least or up to 9 months, at least or up to 12 months, or more than 12 months when stored at 2-8° C. In some such embodiments, the lyophilized formulation retains its cake properties during storage. In some embodiments, the lyophilized formulation is an off-white, uniform and elegant cake without any visible impurities.

In some embodiments, upon reconstitution of a lyophilized formulation following storage, the reconstituted the aqueous formulation is practically free of visible particles. In some embodiments, less than 3% or less than 2% of the DR5-binding polypeptide present in the aqueous formulation is aggregated, and/or less than 1% or less than 0.5% of the DR5-binding polypeptide present in the aqueous formulation is degraded. The presence of aggregated and/or degraded DR5-binding polypeptide may be measured, for example, using size-exclusion chromatography.

Pharmaceutical formulations of a DR5-binding polypeptide as disclosed herein can be presented in a dosage unit form or can be stored in a form suitable for supplying more than one unit dose. A pharmaceutical formulation should be compatible with its intended route of administration. Lyophilized formulations are typically reconstituted in solution prior to administration or use, whereas aqueous formulations may be “ready to use,” meaning that they are administered directly, without being first diluted for example, or can be diluted in saline or another solution prior to use.

Pharmaceutical formulations are preferably sterile. Sterilization can be accomplished by any suitable method, e.g., filtration through sterile filtration membranes. Where the composition is lyophilized, filter sterilization can be conducted prior to or following lyophilization and reconstitution. In various embodiments, lyophilized formulations are provided, which may be reconstituted to form a liquid pharmaceutical formulations provided herein.

### Exemplary Methods of Treating Diseases Using the DR5-Binding Polypeptide Formulations

In some embodiments, methods of treating disease in an individual comprising administering a pharmaceutical formulation comprising a DR5-binding polypeptide are provided. In some embodiments, methods for treating cancer in an individual are provided.

In some embodiments, the method comprises administering to the individual an effective amount of a pharmaceutical formulation comprising a DR5-binding polypeptide provided herein. Such methods of treatment may be in humans or animals. In some embodiments, methods of treating humans are provided. Nonlimiting exemplary cancers that may be treated with DR5-binding polypeptides provided herein include basal cell carcinoma, biliary tract cancer; bladder cancer; bone cancer; brain and central nervous system cancer; breast cancer; cancer of the peritoneum; cervical cancer; choriocarcinoma; chondrosarcoma; Ewing sarcoma; colon and rectum cancer (colorectal cancer); connective tissue cancer; cancer of the digestive system; endometrial cancer; esophageal cancer; eye cancer; cancer of the head and neck; gastric cancer; gastrointestinal cancer; glioblastoma; hepatic carcinoma; hepatoma; intra-epithelial neoplasm; kidney or renal cancer; larynx cancer; liver cancer; lung cancer; small-cell lung cancer; non-small cell lung cancer; adenocarcinoma of the lung; squamous carcinoma of the lung; melanoma; myeloma; neuroblastoma; oral cavity cancer; ovarian cancer; pancreatic cancer, such as pancreatic adenocarcinoma; prostate cancer; retinoblastoma; rhabdomyosarcoma; rectal cancer; cancer of the respiratory system; mesothelioma; salivary gland carcinoma; sarcoma; skin cancer; squamous cell cancer; stomach cancer; testicular cancer; thyroid cancer; uterine or endometrial cancer; cancer of the urinary system; and vulval cancer; lymphoma; Hodgkin's lymphoma; non-Hodgkin's lymphoma; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; chronic lymphocytic leukemia (CLL); acute lymphoblastic leukemia (ALL); Hairy cell leukemia; and chronic myeloblastic leukemia.

The pharmaceutical formulation can be administered as needed to subjects. Determination of the frequency of administration can be made by persons skilled in the art, such as an attending physician based on considerations of the condition being treated, age of the subject being treated, severity of the condition being treated, general state of health of the subject being treated and the like. In some embodiments, an effective dose of a DR5-binding polypeptides is administered to a subject one or more times. In some embodiments, an effective dose of a DR5-binding polypeptides is administered to the subject daily, semiweekly, weekly, every two weeks, once a month, etc. An effective dose of a DR5-binding polypeptides is administered to the subject at least once. In some embodiments, the effective dose of a DR5-binding polypeptides may be administered multiple times, including multiple times over the course of at least a month, at least six months, or at least a year.

In some embodiments, pharmaceutical formulations are administered in an amount effective for treating (including prophylaxis of) cancer. The therapeutically effective amount is typically dependent on the weight of the subject being treated, his or her physical or health condition, the extensiveness of the condition to be treated, or the age of the subject being treated. In general, antibodies may be administered in an amount in the range of about 0.05 mg/kg body weight to about 100 mg/kg body weight per dose. In some embodiments, antibodies may be administered in an amount in the range of about 10 g/kg body weight to about 100 mg/kg body weight per dose. In some embodiments, antibodies may be administered in an amount in the range of about 50 g/kg body weight to about 5 mg/kg body weight per dose. In some embodiments, antibodies may be administered in an amount in the range of about 100 g/kg body weight to about 10 mg/kg body weight per dose. In some embodiments, antibodies may be administered in an amount in the range of about 100 g/kg body weight to about 20 mg/kg body weight per dose. In some embodiments, antibodies may be administered in an amount in the range of about 0.5 mg/kg body weight to about 20 mg/kg body weight per dose. In some embodiments, antibodies may be administered in an amount in the range of about 0.5 mg/kg body weight to about 10 mg/kg body weight per dose. In some embodiments, antibodies may be administered in an amount in the range of about 0.05 mg/kg body weight to about 20 mg/kg body weight per dose. In some embodiments, antibodies may be administered in an amount in the range of about 0.05 mg/kg body weight to about 10 mg/kg body weight per dose. In some embodiments, antibodies may be administered in an amount in the range of about 5 mg/kg body weight or lower, for example less than 4, less than 3, less than 2, or less than 1 mg/kg of the antibody.

In some embodiments, DR5-binding polypeptides can be administered in vivo by various routes, including, but not limited to, intramuscular, intravenous, intra-arterial, parenteral, intraperitoneal or subcutaneous. The appropriate formulation and route of administration may be selected according to the intended application.

### Combination Therapy

DR5-binding polypeptides can be administered alone or in combination with other modes of treatment, such as other anti-cancer agents. They can be provided before, substantially contemporaneous with, or after other modes of treatment (i.e., concurrently or sequentially). In some embodiments, the method of treatment described herein can further include administering: radiation therapy, chemotherapy, vaccination, targeted tumor therapy, CAR-T therapy, oncolytic virus therapy, cancer immunotherapy, cytokine therapy, surgical resection, chromatin modification, ablation, cryotherapy, an antisense agent against a tumor target, a siRNA agent against a tumor target, a microRNA agent against a tumor target or an anti-cancer/tumor agent, or a biologic, such as an antibody, cytokine, or receptor extracellular domain-Fc fusion.

In some embodiments, a DR5-binding polypeptide provided herein is given concurrently with a second therapeutic agent, for example, a PD-1 or PD-L1 therapy. Examples of PD-1/PD-L1 therapy include nivolumab (BMS); pidilizumab (CureTech, CT-011), pembrolizumab (Merck); durvalumab (Medimmune/AstraZeneca); atezolizumab (Genentech/Roche); avelumab (Pfizer); AMP-224 (Amplimmune); BMS-936559; AMP-514 (Amplimmune); MDX-1105 (Merck); TSR-042 (Tesaro/AnaptysBio, ANB-011); STI-A1010 (Sorrento Therapeutics); STI-A1110 (Sorrento Therapeutics); and other agents that are directed against programmed death-1 (PD-1) or programmed death ligand 1 (PD-L1).

In some embodiments, a DR5-binding polypeptide provided herein is given concurrently with an immune stimulatory agent, for example, an agonist of a member of the Tumor Necrosis Factor Receptor Super Family (TNFRSF) or a member the B7 family. Nonlimiting examples of immune stimulatory TNFRSF members include OX40, GITR, 41BB, CD27, and HVEM. Nonlimiting examples of B7 family members include CD28 and ICOS. Thus, in some embodiments, a CD8-binding polypeptide provided herein is given concurrently with an agonist, such as an agonist antibody, of OX40, GITR, 41BB, CD27, HVEM, CD28, and/or ICOS.

In some embodiments, a DR5-binding polypeptide provided herein is given concurrently with CAR-T (chimeric antigen receptor T-cell) therapy, oncolytic virus therapy, cytokine therapy, and/or agents that target other checkpoint molecules, such as VISTA, gpNMB, B7H3, B7H4, HHLA2, CTLA4, TIGIT, etc.

### Kits

Also provided are articles of manufacture and kits that include any of the formulations provided herein and suitable packaging. In some embodiments, the invention includes a kit with (i) a formulation comprising a DR5-binding polypeptide, and (ii) instructions for using the kit to administer the formulation to an individual.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Also provided are unit dosage forms comprising the compositions described herein. These unit dosage forms can be stored in a suitable packaging in single or multiple unit dosages and may also be further sterilized and sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the antibodies generally include information as to dosage, dosing schedule, and route of administration for the intended treatment or industrial use. The kit may further comprise a description of selecting an individual suitable or treatment.

The containers may be unit doses, bulk packages (e.g., multi-dose packages) or sub-unit doses. For example, kits may also be provided that contain sufficient dosages of molecules disclosed herein to provide effective treatment for an individual for an extended period, such as about any of a week, 2 weeks, 3 weeks, 4 weeks, 6 weeks, 8 weeks, 3 months, 4 months, 5 months, 6 months, 7 months, 8 months, 9 months, or more. Kits may also include multiple unit doses of molecules and instructions for use and packaged in quantities sufficient for storage and use in pharmacies, for example, hospital pharmacies and compounding pharmacies. In some embodiments, the kit includes a dry (e.g., lyophilized) composition that can be reconstituted, resuspended, or rehydrated to form generally a stable aqueous solution of antibody.

### EXAMPLES

The examples discussed below are intended to be purely exemplary of the invention and should not be considered to limit the invention in any way. The examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Initial Lyophilized INBRX-109 Formulation Development

The Phase 1 formulation of INBRX-109 contained 20 mg/mL INBRX-109, 20 mM sodium acetate, 263 mM of sucrose, and 0.20% poloxamer 188 at pH 5.0. When stored in a liquid state, the formulation was found to develop visible proteinaceous particles after 6 months at 5° C. The clinical product was therefore frozen to prevent particle formation. A lyophilized formulation was therefore developed for the next clinical phase.

The goal was to develop a lyophilized formulation that is practically free of visible particles and show a minimal increase in high molecular weight (HMW) species by size-exclusion (SEC) chromatography, both after reconstitution and during a 2 year storage at 5° C.

While the Phase 1 formulation contained 20 mM acetate, histidine is more compatible with the freeze-drying process, and accordingly, lyophilized formulations comprising histidine were initially developed and tested. The initial lyophilized formulations included 20 mM histidine buffer, pH 6.0. Formulations at 20 mg/ml INBRX-109 contained 0.2% poloxamer P188, and formulations at 40 mg/ml INBRX-109 contained 0.4% poloxamer P188. Three different lyoprotectants, sucrose, trehalose, or sucrose and mannitol, were tested at varying % (w/v) concentrations. The formulations were filled 2 ml into glass vials. Changes in soluble aggregates after one freeze/thaw cycle and post-lyophilization, as well as after storage of the lyophilized formulation for 1 month at 40° C. or 50° C., were determined by SEC analysis using a TSK gel G3000SWxl column (30 cm×7.8 mm, 5 μm) and an Agilent HPLC. The mobile phase was 50 mM sodium phosphate buffer, 300 mM NaCl, pH 6.8. The flow rate was 1.0 mL/min. Samples were diluted to 1 mg/mL, sterile filtered with a 0.22 μm PES membrane in case of any visible particles, injected on the HPLC column at a volume of 10 μL and detected at 280 nm. The results are shown in Table 1.

TABLE 1

| | Protein | | | | Liquid | after | Post | lyo 40 C. | Lyo 50 C. | change at | change |
|---|---|---|---|---|---|---|---|---|---|---|---|
| Formulation | (mg/ml) | Sucrose | Trehalose | Mannitol | initial | F/T | Lyo | 1 mo | 1 mo | 40 C. 1 mo | 50 C. 1 mo |
| 1A | 20 | | 4 | | 0.9 | 0.9 | 1.1 | 1.2 | 1.7 | 0.1 | 0.6 |
| 2A | 20 | | 6 | | 0.9 | 0.9 | 1.0 | 1.1 | 1.4 | 0.1 | 0.4 |
| 3A | 20 | | 8 | | 0.9 | 0.9 | 1.2 | 1.1 | 1.3 | −0.1 | 0.1 |
| 4A | 20 | 4 | | | 0.9 | 0.9 | 0.9 | 1.0 | 1.2 | 0.1 | 0.3 |
| 5A | 20 | 6 | | | 0.9 | 0.9 | 0.9 | 1.0 | 1.1 | 0.1 | 0.2 |
| 6A | 20 | 8 | | | 0.9 | 0.9 | 0.9 | 1.0 | 1.0 | 0.1 | 0.1 |
| 7A | 20 | 2 | | 4 | 1.0 | 1.2 | 0.9 | 1.6 | 2.3 | 0.7 | 1.4 |
| 8A | 20 | | | | 0.9 | 0.9 | 0.9 | 4.4 | 5.7 | 3.5 | 4.8 |
| 1B | 40 | | 4 | | 1.0 | 1.0 | 0.9 | 1.8 | 2.9 | 0.9 | 2.0 |
| 2B | 40 | | 6 | | 1.0 | 1.0 | 1.0 | 1.5 | 2.1 | 0.5 | 1.1 |
| 3B | 40 | | 8 | | 1.0 | 1.0 | 1.0 | 1.3 | 1.8 | 0.3 | 0.8 |
| 4B | 40 | 4 | | | 1.0 | 1.0 | 1.1 | 1.4 | 2.0 | 0.3 | 0.9 |
| 5B | 40 | 6 | | | 1.1 | 1.0 | 1.0 | 1.2 | 1.5 | 0.2 | 0.5 |
| 6B | 40 | 8 | | | 1.0 | 1.0 | 1.0 | 1.1 | 1.3 | 0.1 | 0.3 |
| 7B | 40 | 2 | | 4 | 1.1 | 1.0 | 1.0 | 2.2 | 3.4 | 1.2 | 2.4 |
| 8B | 40 | | | | 1.2 | 1.0 | 1.0 | 6.2 | 10.3 | 5.2 | 9.3 |

As shown in Table 1, sucrose was superior to trehalose as a lyoprotectant in this formulation (compare IA-3A vs 4A-6A; 1B-3B vs 4B-6B), and higher concentrations of sucrose were more stabilizing (compare 4A to 6A; 4B to 6B).

### Example 2: Further Lyophilized INBRX-109 Formulation Development

A series of further formulations were tested, with 50 mg/mL INBRX-109, 10 mM histidine, pH 6, and 0.2% poloxamer P188. All formulations in this series also included 5 mM methionine, and either trehalose or sucrose, as shown in Table 2.

TABLE 2

| Number | Protein | Buffer | Methionine | Trehalose | Sucrose | Proline | Poloxamer |
|---|---|---|---|---|---|---|---|
| 1 | 50 mg/mL | 10 mM histidine, pH 6.0 | 5 mM | 260 mM | — | — | 0.2% |
| 2 | 50 mg/mL | 10 mM histidine, pH 6.0 | 5 mM | 230 mM | — | — | 0.2% |
| 3 | 50 mg/mL | 10 mM histidine, pH 6.0 | 5 mM | 200 mM | — | — | 0.2% |
| 4 | 50 mg/mL | 10 mM histidine, pH 6.0 | 5 mM | 170 mM | — | — | 0.2% |
| 5 | 50 mg/mL | 10 mM histidine, pH 6.0 | 5 mM | 260 mM | — | — | 0.8% |
| 6 | 50 mg/mL | 10 mM histidine, pH 6.0 | 5 mM | 230 mM | — | — | 0.8% |
| 7 | 50 mg/mL | 10 mM histidine, pH 6.0 | 5 mM | — | 260 mM | — | 0.2% |
| 8 | 50 mg/mL | 10 mM histidine, pH 6.0 | 5 mM | — | 230 mM | — | 0.2% |
| 9 | 50 mg/mL | 10 mM histidine, pH 6.0 | 5 mM | — | 200 mM | — | 0.2% |
| 10 | 50 mg/mL | 10 mM histidine, pH 6.0 | 5 mM | 200 mM | — | 60 mM | 0.2% |

Prior to lyophilization, each formulation in Table 2 was assayed for the appearance of visible particles, and by SEC. After lyophilization, both at time 0 and after 1 week storage at 40° C., the formulations were assayed for cake appearance and reconstitution time, and the reconstituted formulations were assayed for the appearance of visible particles, and by SEC and HIAC. The formulations were examined against a black and white background using a YB-2 lightbox for the appearance test. The lyophilization cycle used for this study is shown in Table 3.

TABLE 3

| | | Shelf temp | Ramp time | Hold time | Chamber pressure |
|---|---|---|---|---|---|
| Step | | (° C.) | (min) | (min) | (mbar) |
| Freezing | 1 | 5 | 20 | 120 | 1 Atm |
| | 2 | −45 | 50 | 120 | 1 Atm |
| | 3 | −7 | 38 | 180 | 1 Atm |
| | 4 | −45 | 38 | 180 | 1 Atm |
| Primary | 5 | −25 | 25 | 3200 | 0.1 |
| drying | 6 | −25 | 0 | 600 | 0.1 |
| Second | 7 | 35 | 360 | 600 | 0.1 |
| drying | | | | | |

The results of the appearance test are shown in Table 4. So=slightly opalescent; FVP=free of visible particles.

TABLE 4

| | | | Lyo, T0 | Lyo, 40C-1W |
|---|---|---|---|---|
| | | | After | After |
| No. | Formulation | Prelyo | Recon. | Recon. |
| F1 | 9.0% Tre. + 0.2% Polo | So, FVP | So, FVP | So, FVP |
| F2 | 8.0% Tre. + 0.2% Polo | So, FVP | So, FVP | So, FVP |
| F3 | 7.0% Tre. + 0.2% Polo | So, FVP | So, FVP | So, FVP |
| F4 | 6.0% Tre. + 0.2% Polo | So, FVP | So, FVP | So, FVP |
| F5 | 9.0% Tre. + 0.8% Polo | So, FVP | So, FVP | So, FVP |
| F6 | 8.0% Tre. + 0.8% Polo | So, FVP | So, FVP | So, FVP |
| F7 | 9.0% Suc. + 0.2% Polo | So, FVP | So, FVP | So, FVP |
| F8 | 8.0% Suc. + 0.2% Polo | So, FVP | So, FVP | So, FVP |
| F9 | 7.0% Suc. + 0.2% Polo | So, FVP | So, FVP | So, FVP |
| F10 | 7.0% Tre. + 60 mM | So, FVP | So, FVP | So, FVP |
| | Proline + 0.2% Polo | | | |

The appearance of all formulations was slightly opalescent and free of visible particles, before and after lyophilization and after 1 month storage in the lyophilized state.

As shown in Table 5, SEC analysis of the lyophilized formulations at TO showed a minimal increase after lyophilization of all formulations. There was no change in W after 1 week storage at 40° C. in the sucrose containing formulations, however a loss of SEC column performance during analysis of the trehalose formulations (F1 through F75) at the 1 week timepoint may have resulted in an apparent increase in HMW of these samples. Formulations containing poloxamer within a concentration range of 0.2% to 0.8% showed a similar change in HMW after storage (F2 vs F6).

TABLE 5

| | | | | | | | | | | Change |
|---|---|---|---|---|---|---|---|---|---|---|
| | Prelyo | | | Lyo-T0 | | | Lyo- 40 C.-1 W | | | HMW |
| No. | Main | HMW | LMW | Main | HMW | LMW | Main | HMW | LMW | 40 C. - T0 |
| F1 | 98.6 | 1.2 | 0.2 | 98.5 | 1.4 | 0.1 | 98.4 | 1.5 | 0.1 | 0.1 |
| F2 | 98.7 | 1.2 | 0.1 | 98.5 | 1.4 | 0.1 | 98.4 | 1.5 | 0.1 | 0.1 |
| F3 | 98.7 | 1.2 | 0.1 | 98.5 | 1.4 | 0.1 | 98.3 | 1.6 | 0.1 | 0.2 |
| F4 | 98.7 | 1.2 | 0.1 | 98.5 | 1.4 | 0.1 | 98.2 | 1.7 | 0.1 | 0.3 |
| F5 | 98.6 | 1.2 | 0.2 | 98.5 | 1.3 | 0.1 | 98.4 | 1.5 | 0.2 | 0.2 |
| F6 | 98.6 | 1.2 | 0.2 | 98.5 | 1.3 | 0.1 | 98.6 | 1.3 | 0.1 | 0.0 |
| F7 | 98.7 | 1.2 | 0.1 | 98.6 | 1.3 | 0.1 | 98.7 | 1.2 | 0.1 | −0.1 |
| F8 | 98.6 | 1.2 | 0.2 | 98.5 | 1.3 | 0.1 | 98.7 | 1.2 | 0.1 | −0.1 |
| F9 | 98.6 | 1.2 | 0.2 | 98.5 | 1.3 | 0.1 | 98.6 | 1.2 | 0.1 | −0.1 |
| F10 | 98.6 | 1.2 | 0.1 | 98.5 | 1.4 | 0.1 | 98.6 | 1.3 | 0.1 | −0.1 |

To semi-quantify the relative cake appearance, a “visual appearance” score was developed, where a score of “0” is a cake with very poor appearance (collapse, cracking, meltback, etc.) and a score of “10” is an elegant solid cake with no detrimental appearance (solid cake with no cracking, meltback, collapse, etc.). Table 6 shows cake appearance and visual appearance score at time 0:

TABLE 6

| | | Visual Appearance |
|---|---|---|
| Formulation | Cake Appearance (T = 0) | Score |
| F1 | White to off-white cake, slight glaze | 6/10 |
| | on upper surface | |
| F2 | White to off-white cake, medium | 6/10 |
| | glaze on upper surface | |
| F3 | White to off-white cake, medium | 6/10 |
| | glaze on upper surface, slight | |
| | collapse on top | |
| F4 | White to off-white cake, medium | 4/10 |
| | glaze on upper surface, moderate | |
| | collapse on top | |
| F5 | White to off-white, less dense cake, | 4/10 |
| | medium glaze on top, bubbles on top | |
| F6 | White to off-white cake, medium | 6/10 |
| | glaze on upper surface | |
| F7 | White to off-white cake, minimal | 8/10 |
| | glaze or collapse | |
| F8 | White to off-white cake, minimal | 8/10 |
| | glaze and no collapse or pullback | |
| F9 | White to off-white cake, moderate | 5/10 |
| | glaze and slight pullback with | |
| | cracked cake on top | |
| F10 | White to off-white cake, medium | 6/10 |
| | glaze on upper surface | |

The appearance of each formulation after reconstitution with water to 50 mg/ml INBRX-109 and storage at 25° C. for up to 30 hours was then determined. The results are shown in Table 7. PO=particles observed; FVP=free of visible particles; (+)=more particles observed; (−)=fewer particles observed.

TABLE 7

25 C.

| | T0 | 4 H | 8 H | 18 H | 24 H | 30 H |
|---|---|---|---|---|---|---|
| F1 | FVP | PO+ | PO+ | PO+ | PO+ | PO+ |
| F3 | FVP | FVP | FVP | FVP | PO | PO |
| F4 | FVP | FVP | FVP | FVP | PO | PO |
| F5 | FVP | PO | PO | PO | PO | PO |
| F6 | FVP | PO− | PO− | PO− | PO− | PO |
| F7 | FVP | FVP | FVP | FVP | PO | PO |
| F8 | FVP | FVP | FVP | FVP | FVP | PO |
| F9 | FVP | FVP | FVP | FVP | FVP | FVP |
| F10 | FVP | FVP | FVP | FVP | FVP | PO |

It was found that trehalose-containing formulations (F1 through F4) were more likely to form particles, showing the presence of visible particles at just 4 hours storage (F1) and by 24 hours storage for all trehalose-containing formulations. It was also found that the concentration of poloxamer did not change the time to form particles, as both F5 and F6 (0.8% poloxamer) showed visible particles after 4 hours, the same as F1 (0.2% poloxamer). Sucrose containing formulations were superior to trehalose in the time to particle formation. Formulation F7 (260 mM sucrose) formed particles at 24 hours vs 4 hours for F1 (260 mM trehalose), while F9 (200 mM sucrose) was particle-free at 30 hours, compared to F3 (200 mM trehalose), which was particle-free only to the 18 hour timepoint.

### Example 3: Formulation Stability Prior to Lyophilization

Formulations were assessed for physical stability (aggregation by SEC and appearance of visible particles) during a short-term storage as a liquid drug substance, prior to lyophilization. A new set of formulations were prepared at 25 or 50 mg/ml protein, in 20 mM histidine HCl buffer pH 6.0, with either poloxamer or polysorbate 80 as non-ionic surfactants. A control formulation in 20 mM sodium acetate pH 5.0 buffer with 8% sucrose with poloxamer was also prepared and tested. The formulations were stored at 25° C. for up to 3 days and observed for particle formation. The formulations are shown in Table 8, and the results are shown in Table 9. FVP=free of visible particles.

TABLE 8

| | DS Conc. | Buffer | Excipient | Surfactant | pH |
|---|---|---|---|---|---|
| F11 | 50 mg/mL | 20 mM | 8% Sucrose | 0.2% Polo188 | 5.0 |
| (con- | | Ace | | | |
| trol) | | | | | |
| F12 | 25 mg/mL | 20 mM His | 8% Sucrose | 0.2% Polo188 | 6.0 |
| F13 | 50 mg/mL | 20 mM His | 8% Sucrose | 0.2% Polo188 | 6.0 |
| F14 | 25 mg/mL | 20 mM His | 8% Sucrose | 0.06% PS80 | 6.0 |
| F15 | 50 mg/mL | 20 mM His | 8% Sucrose | 0.06% PS80 | 6.0 |

TABLE 9

25 C.

| | T0 | 6 H | 18 H | 24 H | 2 D | 3 D |
|---|---|---|---|---|---|---|
| F11 | FVP | FVP | FVP | FVP | FVP | FVP |
| F12 | FVP | FVP | FVP | FVP | FVP | FVP |
| F13 | FVP | FVP | FVP | FVP | FVP | FVP |
| F14 | FVP | FVP | FVP | FVP | FVP | FVP |
| F15 | FVP | FVP | FVP | FVP | FVP | FVP |

As shown in Table 9, when stored at 25° C., these formulations were particle-free for up to 3 days, showing that histidine formulations with sucrose and either poloxamer or polysorbate 80 were as stable as the control formulation, which comprised acetate. Both 20 and 50 mg/ml formulations were stable under these conditions.

The change in high molecular weight (HMW) aggregates was also assayed by SEC after 1 day and 3 days at 25° C. As shown in Table 10, HMW aggregate formation was similar between formulations containing sodium acetate pH 5.0 buffer vs histidine HCl pH 6.0 buffer when stored for up to 3 days at 25° C. in the liquid state. As expected, 50 mg/ml formulations had a higher rate of aggregation (bold; F11, F13, and F15) compared to 20 mg/ml formulations. LMW=low molecular weight species.

TABLE 10

| | T0 % | | | 25 C.-1 D % | | | 25 C.-3 D % | | |
|---|---|---|---|---|---|---|---|---|---|
| | Main | HMW | LMW | Main | HMW | LMW | Main | HMW | LMW |
| F11 | 98.7 | 1.2 | 0.1 | 98.6 | 1.3 | 0.1 | 98.4 | 1.5 | 0.1 |
| F12 | 98.9 | 1.0 | 0.1 | 98.9 | 1.0 | 0.1 | 98.8 | 1.1 | 0.1 |
| F13 | 98.8 | 1.1 | 0.1 | 98.7 | 1.2 | 0.1 | 98.5 | 1.4 | 0.1 |
| F14 | 98.9 | 1.1 | 0.1 | 98.9 | 1.0 | 0.1 | 98.8 | 1.1 | 0.1 |
| F15 | 98.8 | 1.2 | 0.1 | 98.6 | 1.3 | 0.1 | 98.5 | 1.4 | 0.1 |

### Example 4: Varying Concentrations of Sucrose and INBRX-109

Additional formulations were developed to optimize the concentration of sucrose and evaluate a 25 mg/ml protein concentration. These formulations also included methionine, which may act as a stabilizer. The formulations are shown in Table 11.

TABLE 11

| | DS Conc. | Buffer | Excipient | Methionine | Surfactant | pH |
|---|---|---|---|---|---|---|
| F16 | 50 mg/ml | 10 mM His | 8% Sucrose | 5 mM | 0.2% Polo188 | 6.0 |
| F17 | 50 mg/ml | 10 mM His | 9% Sucrose | 5 mM | 0.2% Polo188 | 6.0 |
| F18 | 25 mg/ml | 10 mM His | 9% Sucrose | 5 mM | 0.2% Polo188 | 6.0 |

The three formulations were assayed for appearance pre-lyophilization, at time 0 post-lyophilization (assayed after reconstitution), after 4 weeks storage of lyophilized formulation at 40° C. (assayed after reconstitution), after three freeze/thaw cycles of the liquid formulation, and after 2 weeks or 4 weeks storage of the liquid formulation at 25° C. The results are shown in Table 12. SO—slightly opalescent; FVP=free of visible particles.

TABLE 12

| | | Appearance | | | | | | |
|---|---|---|---|---|---|---|---|---|
| | | | | Lyo-40 C.- | FT-3x | 5 C.-4 W | 25 C.-2 W | 25 C.-4 W |
| No. | Form. | Prelyo-T0 | Lyo-T0 | 4 W | (liquid) | (liquid) | (liquid) | (liquid) |
| F16 | 50 mg/mL, | SO, FVP | SO, FVP | SO, FVP | SO, FVP | SO, FVP | SO, FVP | SO, FVP |
| | 8% suc. | | | | | | | |
| F17 | 50 mg/mL, | SO, FVP | SO, FVP | SO, FVP | SO, FVP | SO, FVP | SO, FVP | SO, FVP |
| | 9% suc. | | | | | | | |
| F18 | 25 mg/mL, | SO, FVP | SO, FVP | SO, FVP | SO, FVP | SO, FVP | SO, FVP | SO, FVP |
| | 9% suc. | | | | | | | |

As shown in Table 12, all three formulations were free of visible particles when viewed under a standard inspection procedure before and after lyophilization, after 4 weeks storage at 40° C. in the lyophilized state, after three cycles of freeze-thaw, and after 2 and 4 weeks storage at 25° C. in liquid form. When viewed using a closer inspection procedure (USP <790>), formulations with 9% sucrose (F17 and F18) showed ˜-10 very small particles after lyophilization, at time 0. All 3 formulations showed these small particles upon close inspection after the longest stress condition of 4 weeks at 25° C. in liquid form.

The visual appearance of all lyophilized formulations was acceptable, appearing as white to off-white solid cakes with no evidence of physical collapse, shrinkage or cracking.

The formulations were also tested for lyophilized moisture content, reconstitution time, and retained potency after 4 weeks storage as a lyophilized formulation at 40° C., 4 weeks storage as a liquid formulation at 25° C., and after three freeze/thaw cycles. As shown in Table 13, all three formulations were within acceptable ranges.

TABLE 13

| | | | | Potency (cell based) | | |
|---|---|---|---|---|---|---|
| | | Moisture content | Recon time | | FT-3x | 25 C.-4 W |
| No. | Form. | Lyo-T0 | Lyo-T0 | Lyo-40 C.-4 W | (liquid) | (liquid) |
| F16 | 50 mg/mL, | 0.49% | 2′ 25″ | 111% | / | 126% |
| | 8% suc. | | | | | |
| F17 | 50 mg/mL, | 0.50% | 2′ 35″ | 117% | 123% | 128% |
| | 9% suc. | | | | | |
| F18 | 25 mg/mL, | 0.83% | 2′ 01″ | 110% | 117% | 111% |
| | 9% suc. | | | | | |

The formulations were also assayed for HMW aggregate formation by SEC. All three formulations were found to be stable, as measured by HMW aggregate formation, prior to lyophilization, after lyophilization, and after 4 weeks storage as lyophilized formulation at 40° C. The results are shown in Table 14.

TABLE 14

| | | Prelyo-T0 | | | | Lyo-T0 | | | | Lyo-40 C.-4 W | | | |
|---|---|---|---|---|---|---|---|---|---|---|---|---|---|
| | | % | | | | % | | | | % | | | |
| No. | Form. | Main | HHMW | HMW | LMW | Main | HHMW | HMW | LMW | Main | HHMW | HMW | LMW |
| F16 | 50 mg/mL, | 98.7 | 0.03 | 1.2 | 0.1 | 98.5 | 0.03 | 1.4 | 0.1 | 98.6 | 0.05 | 1.3 | 0.1 |
| | 8% suc. | | | | | | | | | | | | |
| F17 | 50 mg/mL, | 98.7 | 0.05 | 1.2 | 0.1 | 98.6 | 0.04 | 1.3 | 0.1 | 98.6 | 0.04 | 1.3 | 0.1 |
| | 9% suc. | | | | | | | | | | | | |
| F18 | 25 mg/mL, | 98.8 | 0.01 | 1.1 | 0.1 | 98.7 | 0.03 | 1.2 | 0.1 | 98.7 | 0.03 | 1.2 | 0.1 |
| | 9% suc. | | | | | | | | | | | | |

As shown in Table 15, in the liquid state, the formulations at 50 mg/ml showed a slight increase in HMW aggregation measured by SEC after 4 weeks at 25° C., with no change after three freeze/thaw cycles or 4 weeks storage at 25° C.

TABLE 15

| | | FT-3x (liquid) | | | | SC-4 W (liquid) | | | |
|---|---|---|---|---|---|---|---|---|---|
| | | % | | | | % | | | |
| No. | Form. | Main | HHMW | HMW | LMW | Main | HHMW | HMW | LMW |
| F16 | 50 mg/mL, | 98.7 | 0.03 | 1.2 | 0.1 | 98.6 | 0.03 | 1.3 | 0.1 |
| | 8% suc. | | | | | | | | |
| F17 | 50 mg/mL, | 98.7 | 0.04 | 1.2 | 0.1 | 98.6 | 0.03 | 1.3 | 0.1 |
| | 9% suc. | | | | | | | | |
| F18 | 25 mg/mL, | 98.8 | 0.02 | 1.1 | 0.1 | 98.8 | 0.01 | 1.1 | 0.1 |
| | 9% suc. | | | | | | | | |

| | 25 C.-2 W (liquid) | | | | 25 C.-4 W (liquid) | | | |
|---|---|---|---|---|---|---|---|---|
| | % | | | | % | | | |
| No. | Main | HHMW | HMW | LMW | Main | HHMW | HMW | LMW |
| F16 | 98.3 | 0.04 | 1.6 | 0.2 | 97.9 | 0.05 | 1.8 | 0.2 |
| F17 | 98.3 | 0.03 | 1.6 | 0.2 | 98.0 | 0.04 | 1.8 | 0.2 |
| F18 | 98.7 | 0.03 | 1.1 | 0.2 | 98.5 | 0.03 | 1.2 | 0.2 |

The formulations were then assayed for the presence and number of sub-visible particles per milliliter using High Accuracy Fluid Particle Counting (HIAC) before lyophilization, after lyophilization at time 0, and after storage of the lyophilized formulation at 40° C. for 4 weeks. Particles were measured and counted using a HIAC instrument, with the reported value as the average of triplicate measurements of each sample. The results are shown in Table 16.

TABLE 16

| | | Prelyo-T0 | | | Lyo-T0 | | | Lyo-40 C.-4 W | | |
|---|---|---|---|---|---|---|---|---|---|---|
| | | #/mL | | | #/mL | | | #/mL | | |
| No. | Form. | ≥2 μm | ≥10 μm | ≥25 μm | ≥2 μm | ≥10 μm | ≥25 μm | ≥2 μm | ≥10 μm | ≥25 μm |
| F16 | 50 mg/mL, | 142 | 5 | 0 | 979 | 24 | 0 | 3060 | 24 | 0 |
| | 8% suc. | | | | | | | | | |
| F17 | 50 mg/mL, | 282 | 14 | 0 | 1258 | 19 | 2 | 3338 | 37 | 0 |
| | 9% suc. | | | | | | | | | |
| F18 | 25 mg/mL, | 94 | 5 | 0 | 6309 | 29 | 0 | 332 | 5 | 0 |
| | 9% suc. | | | | | | | | | |

All three formulations showed very low sub-visible particles per milliliter after lyophilization and storage in the lyophilized state after 4 weeks at 40° C. (Acceptable limits for subvisible particles are <6000 particles/container (1000 particles/mL) for ≥10 μm particles, and <600 particles/container (100 particles/mL) for ≥25 μm size).

Table 17 shows the number of subvisible particles in the liquid formulations under various conditions.

TABLE 17

| | | Prełyo-T0 | | | FT-3x (liquid) | | | 5 C.-4 W (liquid) | | |
|---|---|---|---|---|---|---|---|---|---|---|
| | | #/mL | | | #/mL | | | #/mL | | |
| No. | Form. | ≥2 μm | ≥10 μm | ≥25 μm | ≥2 μm | ≥10 μm | ≥25 μm | ≥2 μm | ≥10 μm | ≥25 μm |
| F16 | 50 mg/mL, | 142 | 5 | 0 | 1182 | 40 | 2 | 1579 | 29 | 3 |
| | 8% suc. | | | | | | | | | |
| F17 | 50 mg/mL, | 282 | 14 | 0 | 159 | 13 | 0 | 1298 | 30 | 3 |
| | 9% suc. | | | | | | | | | |
| F18 | 25 mg/mL, | 94 | 5 | 0 | 845 | 28 | 2 | 575 | 23 | 0 |
| | 9% suc. | | | | | | | | | |

| | 25 C.-2 W (liquid) | | | 25 C.-4 W (liquid) | | |
|---|---|---|---|---|---|---|
| | #/mL | | | #/mL | | |
| No. | ≥2 μm | ≥10 μm | ≥25 μm | ≥2 μm | ≥10 μm | >25 μm |
| F16 | 1040 | 60 | 3 | 793 | 42 | 2 |
| F17 | 1060 | 35 | 3 | 1233 | 79 | 3 |
| F18 | 803 | 47 | 3 | 625 | 29 | 2 |

Subvisible particles were also within acceptable limits in the liquid formulations under the conditions tested, demonstrating that the formulations showed good physical stability.

The size distribution of the proteins in the formulation (aggregates (HMW), non-reduced monomer (Main), fragments (LMW)) was measured by non-reduced CE-SDS (capillary electrophoresis with SDS denaturant). As shown in Table 18, the size distribution was found to be essentially unchanged after lyophilization, and after storage of the lyophilized formulations for 4 weeks at 40° C., indicating the protein does not physically degrade in these formulations.

TABLE 18

| | | Prelyo-T0 | | | Lyo-T0 | | | Lyo-40 C.-4 W | | |
|---|---|---|---|---|---|---|---|---|---|---|
| | | % | | | % | | | % | | |
| No. | Form. | Main | HMW | LMW | Main | HMW | LMW | Main | HMW | LMW |
| F16 | 50 mg/mL, | 99.4 | 0.2 | 0.4 | 99.7 | 0.0 | 0.3 | 99.7 | 0.0 | 0.3 |
| | 8% suc. | | | | | | | | | |
| F17 | 50 mg/mL, | 99.4 | 0.2 | 0.4 | 99.7 | 0.0 | 0.3 | 99.8 | 0.0 | 0.2 |
| | 9% suc. | | | | | | | | | |
| F18 | 25 mg/mL, | 99.5 | 0.1 | 0.4 | 99.6 | 0.0 | 0.4 | 99.8 | 0.0 | 0.2 |
| | 9% suc. | | | | | | | | | |

The protein was also stable in the three formulations in the liquid state, with little physical degradation under the conditions assayed, as shown in Table 19.

TABLE 19

| | | Prelyo-T0 | | | FT-3x (liquid) | | | 5 C.-4 W (liquid) | | | 25 C.-2 W (liquid) | | | 25 C.-4 W (liquid) | | |
|---|---|---|---|---|---|---|---|---|---|---|---|---|---|---|---|---|
| | | % | | | % | | | % | | | % | | | % | | |
| No. | Form. | Main | HMW | LMW | Main | HMW | LMW | Main | HMW | LMW | Main | HMW | LMW | Main | HMW | LMW |
| F16 | 50 | 99.4 | 0.2 | 0.4 | 99.4 | 0.1 | 0.5 | 99.4 | 0.2 | 0.4 | 98.8 | 0.3 | 0.9 | 98.8 | 0.3 | 0.9 |
| | mg/mL, | | | | | | | | | | | | | | | |
| | 8% suc. | | | | | | | | | | | | | | | |
| F17 | 50 | 99.4 | 0.2 | 0.4 | 99.4 | 0.1 | 0.5 | 99.0 | 0.2 | 0.9 | 98.9 | 0.2 | 0.9 | 98.8 | 0.3 | 0.9 |
| | mg/mL, | | | | | | | | | | | | | | | |
| | 9% suc. | | | | | | | | | | | | | | | |
| F18 | 25 | 99.5 | 0.1 | 0.4 | 99.5 | 0.1 | 0.4 | 99.1 | 0.2 | 0.8 | 99.0 | 0.1 | 0.9 | 98.9 | 0.1 | 0.9 |
| | mg/mL, | | | | | | | | | | | | | | | |
| | 9% suc. | | | | | | | | | | | | | | | |

The charge profile of the protein (% acidic species, % basic species, % monomer) in the formulations was assayed by capillary isoelectric focusing (cIEF). Acidic species have a lower pI (isoelectric point) than the main peak and may include molecules with increased levels of asparagine (Asn) and/or glutamic acid (Gln) deamidation. Basic species have a higher pI relative to the main peak and may include molecules with C-terminal lysine and/or increased succinimide formation at aspartic acid (Asp) residues. As shown in Table 20, the charge profile was essentially unchanged after lyophilization and after storage of the lyophilized formulation at 40° C. for 4 weeks. These results show the formulations protect against chemical modification of the protein under these conditions.

TABLE 20

| | | Prelyo-T0 | | | Lyo-T0 | | | Lyo-40 C.-4 W | | |
|---|---|---|---|---|---|---|---|---|---|---|
| | | % | | | % | | | % | | |
| No. | Form. | Main | Acidic | Basic | Main | Acidic | Basic | Main | Acidic | Basic |
| F16 | 50 mg/mL, | 59.6 | 25.8 | 14.6 | 61.3 | 23.3 | 15.4 | 61.4 | 24.0 | 14.7 |
| | 8% suc. | | | | | | | | | |
| F17 | 50 mg/mL, | 59.8 | 25.5 | 14.6 | 61.2 | 23.2 | 15.6 | 60.9 | 24.3 | 14.9 |
| | 9% suc. | | | | | | | | | |
| F18 | 25 mg/mL, | 59.7 | 25.8 | 14.6 | 61.4 | 23.9 | 14.8 | 60.7 | 24.1 | 15.2 |
| | 9% suc. | | | | | | | | | |

The formulations also did not show significant change in the charge profile of the protein in the liquid state under the conditions tested, as shown in Table 21. Accordingly, the formulations protect against chemical modification of the protein in the liquid state, indicating the formulations are sufficiently stable to permit manufacturing steps prior to lyophilization for the drug product.

TABLE 21

| | | Prelyo-T0 | | | FT-3x (liquid) | | | 5 C.-4 W (liquid) | | | 25 C.-2 W (liquid) | | | 25 C.-4 W (liquid) | | |
|---|---|---|---|---|---|---|---|---|---|---|---|---|---|---|---|---|
| | | % | | | % | | | % | | | % | | | % | | |
| No. | Form. | Main | Acidic | Basic | Main | Acidic | Basic | Main | Acidic | Basic | Main | Acidic | Basic | Main | Acidic | Basic |
| F16 | 50 mg/mL, | 59.6 | 25.8 | 14.6 | 60.8 | 24.6 | 14.7 | 61.3 | 24.4 | 14.3 | 60.1 | 24.9 | 14.9 | 59.8 | 25.6 | 14.7 |
| | 8% suc. | | | | | | | | | | | | | | | |
| F17 | 50 mg/mL, | 59.8 | 25.5 | 14.6 | 60.5 | 25.2 | 14.3 | 60.7 | 24.8 | 14.4 | 60.5 | 25.0 | 14.5 | 59.7 | 26.1 | 14.2 |
| | 9% suc. | | | | | | | | | | | | | | | |
| F18 | 25 mg/mL, | 59.7 | 25.8 | 14.6 | 61.0 | 24.3 | 14.7 | 60.8 | 24.7 | 14.4 | 60.6 | 24.8 | 14.6 | 59.9 | 25.4 | 14.7 |
| | 9% suc. | | | | | | | | | | | | | | | |

Based on the test results, a final formulation of 50 mg/ml INBRX-109, 10 mM histidine HCl, 8% sucrose, and 0.2% L poloxamer 188 pH 6.0 was selected. 8% sucrose was selected over 900 sucrose based on the initial appearance results, in which the 8% sucrose formulation (F8) was particle free for a longer time after reconstitution compared to 90% sucrose (F7).

A batch of the final formulation of INBRX-109 was made and tested for various characteristics under various conditions, as shown in Table 22.

TABLE 22

| | | Lyophilized development batch | | | | |
|---|---|---|---|---|---|---|
| | Drug | | After reconstitution | | Lyo Drug Product | |
| | Substance | T = 0 | 24 hr 5 C. | 24 hr 25 C. | 1 mo 5 C. | 1 mo 25 C. |
| Appearance | free of VP | 1 fiber in 1 | free of VP | 2 fibers in 1 | free of VP | free of VP |
| | (post | vial (n = 10) | | vial; | | |
| | filtration) | | | 1 particle in | | |
| | | | | 1 vial | | |
| | | | | (n = 10) | | |
| Recon Time | — | <4 min | — | — | <4 min | <4 min |
| % moisture | — | 0.56% | — | — | 0.53% | 0.56% |
| SEC monomer | 98.6 | 98.7 | 98.6 | 98.5 | 98.7 | 98.6 |
| SEC aggregate | 1.3 | 1.3 | 1.3 | 1.4 | 1.3 | 1.3 |
| icIEF main peak | — | 68 | 72 | 71 | 71 | 71 |
| NR-CE-SDS | 99.5 | 99.5 | 99.5 | 99.6 | 99.5 | 99.5 |
| monomer | | | | | | |
| Potency | n/a | 95.5% | 98.3% | 98.7% | 99.7% | 92.8% |
| SVP 10 um | — | 1090 | 14 | 11 | 10 | 10 |
| SVP 25 um | — | 23 | 6 | 0 | 0 | 0 |

VP = visible particles

Product quality attributes of the final formulation showed that this formulation prevented the formation of visible particles both after reconstitution and after storage of the lyophilized drug product, and maintained physical and chemical stability and potency of INBRX-109.

### Example 5: Stability of INBRX-109 Drug Product in Formulation

The stability of a batch of INBRX-109 lyophilized drug product manufactured at the pilot scale after storage at several temperatures was assessed. The lyophilized product was reconstituted with sterile water to form an aqueous formulation containing 50 mg/ml INBRX-109, 10 mM histidine HCl, 8% sucrose, and 0.2% poloxamer 188 pH 6.0.

The results of analytical testing of the reconstituted product are shown in Tables 23 and 24. INBRX-109 in the lyophilized drug product retained its physical, chemical (size distribution by SEC, CE-SDS, charge variant profile by iCIEF) and biological properties (potency) after 9 months storage at the intended storage condition of 2-8° C., and over time at 25° C. and 40° C., temperatures that were used to accelerate product degradation. Under these storage conditions, the lyophilized drug product remained practically free of visible particles. In Tables 23 and 24, “SVP” means sub-visible particles, “SEC” means size exclusion chromatography, “cIEF” means capillary isoelectric focusing, “CE-SDS-NR” means capillary electrophoresis-sodium dodecylsulfate-non-reduced, “CE-SDS-R” means capillary electrophoresis-sodium dodecylsulfate-reduced, “#/C” means #particles/container, “HMW” means high molecular weight species, “Main” means main peak, “LMW” means low molecular weight species, “mOsm” means milliosmoles, “PFVP” means practically free of visible particles, and “RH” means relative humidity.

TABLE 23

| | Time | Appearance | SVP by HIAC | | | | | Protein | SEC |
|---|---|---|---|---|---|---|---|---|---|
| | Point | Visible | #/C | #/C | #/C | #/C | | Concentration | % |
| Storage | (Months) | Particles | ≥2 μm | ≥5 μm | ≥10 μm | ≥25 μm | pH | (mg/mL) | HMW |
| 5 ± 3° C. | 0 | PFVP | 1690 | 120 | 14 | 2 | 6.0 | 50.1 | 1.3 |
| | 3 | PFVP | 456 | 27 | 24 | 0 | 6.1 | 50.8 | 1.3 |
| | 6 | PFVP | 792 | 46 | 2 | 0 | 6.1 | 50.5 | 1.3 |
| | 9 | PFVP | 854 | 98 | 8 | 0 | 6.0 | 50.8 | 1.3 |
| 25 ± 2° C. | 0 | PFVP | 1690 | 120 | 14 | 2 | 6.0 | 50.1 | 1.3 |
| 60 ± 5% | 1 | PFVP | 1186 | 138 | 14 | 0 | 6.1 | 51.0 | 1.3 |
| RH | 2 | PFVP | 1596 | 126 | 14 | 0 | 6.1 | 50.8 | 1.3 |
| | 3 | PFVP | 184 | 31 | 56 | 2 | 6.1 | 51.0 | 1.3 |
| | 6 | PFVP | 622 | 24 | 2 | 0 | 6.1 | 51.1 | 1.4 |
| 40 ± 2° C. | 0 | PFVP | 1690 | 120 | 14 | 2 | 6.0 | 50.1 | 1.3 |
| 75 ± 5% | 0.5 | PFVP | 1038 | 98 | 12 | 0 | 6.1 | 51.3 | 1.3 |
| RH | 1 | PFVP | 762 | 100 | 16 | 0 | 6.1 | 51.6 | 1.4 |
| | 2 | PFVP | 728 | 32 | 2 | 0 | 6.1 | 49.9 | 1.4 |
| | 3 | PFVP | 233 | 9 | 6 | 0 | 6.1 | 50.8 | 1.5 |

| | Time | SEC | | cIEF | | | CE-SDS-NR | | |
|---|---|---|---|---|---|---|---|---|---|
| | Point | % | % | % | % | % | % | % | % |
| Storage | (Months) | Main | LMW | Acidic | Main | Basic | LMW | Main | HMW |
| 5 ± 3° C. | 0 | 98.6 | 0.2 | 26.7 | 70.3 | 3.0 | 0.7 | 98.9 | 0.4 |
| | 3 | 98.1 | 0.6 | 26.8 | 70.2 | 3.0 | 0.6 | 99.0 | 0.4 |
| | 6 | 98.5 | 0.2 | 27.1 | 70.0 | 2.9 | 0.7 | 98.9 | 0.4 |
| | 9 | 98.6 | 0.1 | 26.1 | 70.8 | 3.1 | 0.6 | 99.0 | 0.5 |
| 25 ± 2° C. | 0 | 98.6 | 0.2 | 26.7 | 70.3 | 3.0 | 0.7 | 98.9 | 0.4 |
| 60 ± 5% | 1 | 98.5 | 0.2 | 26.5 | 70.4 | 3.1 | 0.6 | 98.9 | 0.5 |
| RH | 2 | 98.6 | 0.1 | 27.7 | 68.8 | 3.5 | 0.6 | 98.7 | 0.7 |
| | 3 | 98.3 | 0.4 | 26.9 | 69.8 | 3.3 | 0.7 | 98.9 | 0.4 |
| | 6 | 98.5 | 0.2 | 26.9 | 69.1 | 3.9 | 0.7 | 98.9 | 0.5 |
| 40 ± 2° C. | 0 | 98.6 | 0.2 | 26.7 | 70.3 | 3.0 | 0.7 | 98.9 | 0.4 |
| 75 ± 5% | 0.5 | 98.5 | 0.2 | 26.2 | 70.2 | 3.6 | 0.5 | 99.0 | 0.5 |
| RH | 1 | 98.4 | 0.2 | 26.4 | 69.4 | 4.2 | 0.6 | 98.9 | 0.4 |
| | 2 | 98.4 | 0.1 | 26.2 | 69.1 | 4.7 | 0.6 | 98.8 | 0.6 |
| | 3 | 97.9 | 0.6 | 26.1 | 68.6 | 5.3 | 0.7 | 98.8 | 0.5 |

TABLE 24

| | Time Point | CE-SDS-R | | | Osmolality | Potency |
|---|---|---|---|---|---|---|
| Storage | (Months) | % LMW | % Main | % HMW | (mOsm/kg) | (% Activity) |
| 5 ± 3° C. | 0 | 2.8 | 95.8 | 1.5 | 293 | 106 |
| | 3 | 3.0 | 95.9 | 1.1 | 294 | 122 |
| | 6 | 2.8 | 95.4 | 1.8 | 300 | 109 |
| | 9 | 2.9 | 95.9 | 1.2 | 297 | 116 |
| 25 ± 2° C. | 0 | 2.8 | 95.8 | 1.5 | 293 | 106 |
| 60 ± 5% | 1 | 2.8 | 95.7 | 1.5 | 296 | 105 |
| RH | 2 | 3.1 | 95.3 | 1.5 | 289 | 105 |
| | 3 | 3.1 | 95.8 | 1.1 | 295 | 122 |
| | 6 | 2.8 | 95.8 | 1.4 | 298 | 98 |
| 40 ± 2° C. | 0 | 2.8 | 95.8 | 1.5 | 293 | 106 |
| 75 ± 5% | 0.5 | 2.8 | 95.4 | 1.8 | 297 | 109 |
| RH | 1 | 2.8 | 95.7 | 1.5 | 300 | 104 |
| | 2 | 2.9 | 95.8 | 1.3 | 291 | 117 |
| | 3 | 3 | 95.9 | 1.1 | 295 | 126 |

The results of analytical testing of the lyophilized product before reconstitution are shown in Table 25. This data shows that the lyophilized drug product retained its stability over time and across several temperatures, as minimal changes were detected in the physical appearance, reconstitution time, and moisture content of the drug product. “RH” means relative humidity, “NT” means not tested, and “Recon. Time” is the time, in seconds, between the addition of the diluent (sterile water for injection) to the product vial and the complete dissolution of the lyophilized solids, as determined visually.

TABLE 25

| | Time | | Recon. | Moisture |
|---|---|---|---|---|
| | Point | | Time | Content |
| Storage | (Months) | Cake Appearance | (s) | (% w/w) |
| 5 ± | 0 | off-white, uniform and elegant | 240 | 0.6 |
| 3° C. | | cake w/o any visible | | |
| | | impurities | | |
| | 3 | off-white, uniform and elegant | NT | 0.6 |
| | | cake w/o any visible | | |
| | | impurities | | |
| | 6 | off-white, uniform and elegant | 240 | 0.6 |
| | | cake w/o any visible | | |
| | | impurities | | |
| | 9 | off-white, uniform and elegant | 240 | 0.7 |
| | | cake w/o any visible | | |
| | | impurities | | |
| 25 ± | 0 | off-white, uniform and elegant | 240 | 0.6 |
| 2° C. | | cake w/o any visible particles | | |
| 60 ± | 1 | off-white, uniform and elegant | 240 | 0.7 |
| 5% RH | | cake w/o any visible particles | | |
| | 2 | off-white, uniform and elegant | 240 | 0.7 |
| | | cake w/o any visible | | |
| | | impurities | | |
| | 3 | off-white, uniform and elegant | NT | 0.8 |
| | | cake w/o any visible | | |
| | | impurities | | |
| | 6 | off-white, uniform and elegant | 240 | 0.8 |
| | | cake w/o any visible | | |
| | | impurities | | |
| 40 ± | 0 | off-white, uniform and elegant | 240 | 0.6 |
| 2° C. | | cake w/o any visible | | |
| | | impurities | | |
| 75 ± | 0.5 | off-white, uniform and elegant | 240 | 0.7 |
| 5% RH | | cake w/o any visible | | |
| | | impurities | | |
| | 1 | off-white, uniform and elegant | 240 | 0.8 |
| | | cake w/o any visible | | |
| | | impurities | | |
| | 2 | off-white, uniform and elegant | 240 | 0.8 |
| | | cake w/o any visible | | |
| | | impurities | | |
| | 3 | off-white, uniform and elegant | 300 | 0.9 |
| | | cake w/o any visible | | |
| | | impurities | | |
| −20 ± | 0 | off-white, uniform and elegant | 240 | 0.6 |
| 5° C. | | cake w/o any visible | | |
| | | impurities | | |
| | 1 | off-white, uniform and elegant | 240 | 0.6 |
| | | cake w/o any visible | | |
| | | impurities | | |

The disclosure may be embodied in other specific forms without departing from the spirit or essential characteristics thereof. The foregoing embodiments are therefore to be considered in all respects illustrative rather than limiting of the disclosure. Scope of the disclosure is thus indicated by the appended claims rather than by the foregoing description, and all changes that come within the meaning and range of equivalency of the claims are therefore intended to be embraced herein.

TABLE of
Certain Sequences

| SEQ ID | | |
|---|---|---|
| NO | Description | Sequence |
| 1 | INBRX-109 | SGLTFPNYGM |
| | CDR1 | |
| 2 | INBRX-109 | AIYWSGGTVY |
| | CDR2 | |
| 3 | INBRX-109 | AVTIRGAATQTWKYDYW |
| | CDR3 | |
| 4 | INBRX-109 | EVQLLESGGGEVQPGGSLRLSCAASGLTFPNYGMGWFRQAPGKEREFVSAIYWS |
| | VHH | GGTVYYAESVKGRFTISRDNAKNTLYLQMSSLRAEDTAVYYCAVTIRGAATQTW |
| | | KYDYWGQGTLVTVKPGG |
| 5 | INBRX-109 | EVQLLESGGGEVQPGGSLRLSCAASGLTFPNYGMGWFRQAPGKEREFVSAIYWS |
| | VHH-linker- | GGTVYYAESVKGRFTISRDNAKNTLYLQMSSLRAEDTAVYYCAVTIRGAATQTW |
| | VHH | KYDYWGQGTLVTVKPGGSGGSEVQLLESGGGEVQPGGSLRLSCAASGLTFPNYG |
| | | MGWFRQAPGKEREFVSAIYWSGGTVYYAESVKGRFTISRDNAKNTLYLQMSSLR |
| | | AEDTAVYYCAVTIRGAATQTWKYDYWGQGTLVTVKPGG |
| 6 | Fc (with | DKTHTCPPCPAPGGPSVFLFPPKPKDTLMISRTPEVTCVVVDVSHEDPEVKFNW |
| | hinge) | YVDGVEVHNAKTKPREEQYNSTYRVVSVLTVLHQDWLNGKEYKCKVSNKALPAP |
| | | IEKTISKAKGQPREPQVYTLPPSRDELTKNQVSLTCLVKGFYPSDIAVEWESNG |
| | | QPENNYKTTPPVLDSDGSFFLYSKLTVDKSRWQQGNVFSCSVMHEALHNHYTQK |
| | | SLSLSPGK |
| 7 | INBRX-109 | EVQLLESGGGEVQPGGSLRLSCAASGLTFPNYGMGWFRQAPGKEREFVSAIYWS |
| | | GGTVYYAESVKGRFTISRDNAKNTLYLQMSSLRAEDTAVYYCAVTIRGAATQTW |
| | | KYDYWGQGTLVTVKPGGSGGSEVQLLESGGGEVQPGGSLRLSCAASGLTFPNYG |
| | | MGWFRQAPGKEREFVSAIYWSGGTVYYAESVKGRFTISRDNAKNTLYLQMSSLR |
| | | AEDTAVYYCAVTIRGAATQTWKYDYWGQGTLVTVKPGGGGDKTHTCPPCPAPGG |
| | | PSVFLFPPKPKDTLMISRTPEVTCVVVDVSHEDPEVKFNWYVDGVEVHNAKTKP |
| | | REEQYNSTYRVVSVLTVLHQDWLNGKEYKCKVSNKALPAPIEKTISKAKGQPRE |
| | | PQVYTLPPSRDELTKNQVSLTCLVKGFYPSDIAVEWESNGQPENNYKTTPPVLD |
| | | SDGSFFLYSKLTVDKSRWQQGNVFSCSVMHEALHNHYTQKSLSLSPGK |
| 8 | Human DR5 | MEQRGQNAPA ASGARKRHGP GPREARGARP GPRVPKTLVL VVAAVLLLVS |
| | (with signal | AESALITQQD LAPQQRAAPQ QKRSSPSEGL CPPGHHISED GRDCISCKYG |
| | peptide, | QDYSTHWNDL LFCLRCTRCD SGEVELSPCT TTRNTVCQCE EGTFREEDSP |
| | amino acids | EMCRKCRTGC PRGMVKVGDC TPWSDIECVH KESGTKHSGE VPAVEETVTS |
| | 1-55) | SPGTPASPCS LSGIIIGVTV AAVVLIVAVF VCKSLLWKKV LPYLKGICSG |
| | | GGGDPERVDR SSQRPGAEDN VLNEIVSILQ PTQVPEQEME VQEPAEPTGV |
| | | NMLSPGESEH LLEPAEAERS QRRRLLVPAN EGDPTETLRQ CFDDFADLVP |
| | | FDSWEPLMRK LGLMDNEIKV AKAEAAGHRD TLYTMLIKWV NKTGRDASVH |
| | | TLLDALETLG ERLAKQKIED HLLSSGKEMY LEGNADSAMS |
| 9 | Mature | ITQQD LAPQQRAAPQ QKRSSPSEGL CPPGHHISED GRDCISCKYG |
| | human DR5 | QDYSTHWNDL LFCLRCTRCD SGEVELSPCT TTRNTVCQCE EGTFREEDSP |
| | | EMCRKCRTGC PRGMVKVGDC TPWSDIECVH KESGTKHSGE VPAVEETVTS |
| | | SPGTPASPCS LSGIIIGVTV AAVVLIVAVF VCKSLLWKKV LPYLKGICSG |
| | | GGGDPERVDR SSQRPGAEDN VLNEIVSILQ PTQVPEQEME VQEPAEPTGV |
| | | NMLSPGESEH LLEPAEAERS QRRRLLVPAN EGDPTETLRQ CFDDFADLVP |
| | | FDSWEPLMRK LGLMDNEIKV AKAEAAGHRD TLYTMLIKWV NKTGRDASVH |
| | | TLLDALETLG ERLAKQKIED HLLSSGKEMY LEGNADSAMS |

1. A pharmaceutical formulation comprising a DR5-binding polypeptide, wherein the formulation comprises 20-70 mg/mL DR5-binding polypeptide, 5-20 mM histidine, 7-10% w/v sucrose, and 0.1-0.8% poloxamer P188 at a pH of 5.3-6.7; and wherein the DR5-binding polypeptide comprises at least one VHH domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3.

2. (canceled)

3. The pharmaceutical formulation of claim 1, wherein the formulation comprises 50 mg/mL DR5-binding polypeptide.

4. (canceled)

5. The pharmaceutical formulation of claim 1, wherein the formulation comprises 10 mM histidine.

6. The pharmaceutical formulation of claim 1, wherein the histidine is histidine HCl.

7. (canceled)

8. The pharmaceutical formulation of claim 1, wherein the formulation comprises 8% sucrose or 9% sucrose.

9. (canceled)

10. The pharmaceutical formulation of claim 1, wherein the formulation comprises 0.2% poloxamer P188.

11. (canceled)

12. The pharmaceutical formulation of claim 1, wherein the formulation comprises 5 mM methionine.

13. (canceled)

14. The pharmaceutical formulation of claim 1, wherein the pH of the formulation is about 6.

15. The pharmaceutical formulation of claim 1, wherein the formulation comprises 50 mg/mL DR5-binding polypeptide, 10 mM histidine HCl, 8% sucrose, and 0.2% poloxamer P188, and wherein the pH of the formulation is about 6.

16. The pharmaceutical formulation of claim 15, wherein the formulation consists essentially of 50 mg/mL DR5-binding polypeptide, 10 mM histidine HCl, 8% sucrose, 0.2% poloxamer P188, and water, and wherein the pH of the formulation is about 6.

17. The pharmaceutical formulation of claim 1, wherein the DR5-binding polypeptide comprises a VHH domain comprising the amino acid sequence of SEQ ID NO: 4.

18. The pharmaceutical formulation of claim 1, wherein the DR5-binding polypeptide comprises an Fc region.

19. The pharmaceutical formulation of claim 18, wherein the Fc region comprises the amino acid sequence of SEQ ID NO: 6.

20. The pharmaceutical formulation of claim 1, wherein the DR5-binding polypeptide has the structure VHH-linker-VHH-linker-Fc.

21. The pharmaceutical formulation of claim 20, wherein the VHH-linker-VHH comprises the amino acid sequence of SEQ ID NO: 5.

22. The pharmaceutical formulation of claim 1, wherein the DR5-binding polypeptide comprises the amino acid sequence of SEQ ID NO: 7.

23. The pharmaceutical formulation of claim 1, wherein the DR5-binding polypeptide consists of the amino acid sequence of SEQ ID NO: 7.

24. A lyophilized formulation comprising a DR5-binding polypeptide, wherein the DR5-binding polypeptide comprises at least one VHH domain comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and wherein upon reconstitution of the lyophilized formulation in water to form an aqueous formulation, the aqueous formulation comprises 20-70 mg/mL DR5-binding polypeptide, 5-20 mM histidine, 7-10% sucrose, and 0.1-0.5% poloxamer P188, pH 5.3-6.7.

47. A lyophilized formulation formed by lyophilizing the pharmaceutical formulation of claim 1.

52. A pharmaceutical formulation formed by reconstituting the lyophilized formulation of claim 24 in water.

53. A method of treating cancer comprising administering to a subject with cancer the pharmaceutical formulation of claim 1.

54. The method of claim 53, wherein the cancer is chondrosarcoma, mesothelioma, colorectal cancer, Ewing sarcoma, or pancreatic adenocarcinoma.

55. The pharmaceutical formulation of claim 22, wherein the formulation comprises 50 mg/mL DR5-binding polypeptide, 10 mM histidine HCl, 8% sucrose, and 0.2% poloxamer P188, and wherein the pH of the formulation is about 6.

56. The pharmaceutical formulation of claim 22, wherein the formulation consists essentially of 50 mg/mL DR5-binding polypeptide, 10 mM histidine HCl, 8% sucrose, 0.2% poloxamer P188, and water, and wherein the pH of the formulation is about 6.

57. The pharmaceutical formulation of claim 23, wherein the formulation comprises 50 mg/mL DR5-binding polypeptide, 10 mM histidine HCl, 8% sucrose, and 0.2% poloxamer P188, and wherein the pH of the formulation is about 6.

58. The pharmaceutical formulation of claim 23, wherein the formulation consists essentially of 50 mg/mL DR5-binding polypeptide, 10 mM histidine HCl, 8% sucrose, 0.2% poloxamer P188, and water, and wherein the pH of the formulation is about 6.


SEQUENCE LISTING (9 sequences)

SEQ ID NO: 1 - protein, 10 aa, Artificial sequence
  INBRX-109 CDR1
SGLTFPNYGM

SEQ ID NO: 2 - protein, 10 aa, Artificial sequence
  INBRX-109 CDR2
AIYWSGGTVY

SEQ ID NO: 3 - protein, 17 aa, Artificial sequence
  INBRX-109 CDR3
AVTIRGAATQTWKYDYW

SEQ ID NO: 4 - protein, 125 aa, Artificial sequence
  INBRX-109 VHH
EVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGG

SEQ ID NO: 5 - protein, 254 aa, Artificial sequence
  INBRX-109 VHH-linker-VHH
EVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGGSGGSEVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGG

SEQ ID NO: 6 - protein, 224 aa, Artificial sequence
  Fc (with hinge)
DKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 480 aa, Artificial sequence
  INBRX-109
EVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGGSGGSEVQLLESGGGEVQPGGSLRLSCAASGLTFPNYGMGWFRQAPGKEREFVSAIYWSGGTVYYAESVKGRFTISRDNAKNTLYLQMSSLRAEDTAVYYCAVTIRGAATQTWKYDYWGQGTLVTVKPGGGGDKTHTCPPCPAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 440 aa, Homo sapiens
MEQRGQNAPAASGARKRHGPGPREARGARPGPRVPKTLVLVVAAVLLLVSAESALITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS

SEQ ID NO: 9 - protein, 385 aa, Homo sapiens
ITQQDLAPQQRAAPQQKRSSPSEGLCPPGHHISEDGRDCISCKYGQDYSTHWNDLLFCLRCTRCDSGEVELSPCTTTRNTVCQCEEGTFREEDSPEMCRKCRTGCPRGMVKVGDCTPWSDIECVHKESGTKHSGEVPAVEETVTSSPGTPASPCSLSGIIIGVTVAAVVLIVAVFVCKSLLWKKVLPYLKGICSGGGGDPERVDRSSQRPGAEDNVLNEIVSILQPTQVPEQEMEVQEPAEPTGVNMLSPGESEHLLEPAEAERSQRRRLLVPANEGDPTETLRQCFDDFADLVPFDSWEPLMRKLGLMDNEIKVAKAEAAGHRDTLYTMLIKWVNKTGRDASVHTLLDALETLGERLAKQKIEDHLLSSGKFMYLEGNADSAMS